(19) United States
(12) Patent Application Publication
(10) Pub. No.: US 2010/0249402 A1
(43) Pub. Date: Sep. 30, 2010

(54) NOVEL BENZAMIDINE DERIVATIVES, PROCESS FOR THE PREPARATION THEREOF AND PHARMACEUTICAL COMPOSITION FOR PREVENTING OR TREATING OSTEPOROSIS COMPRISING THE SAME

(75) Inventors: Jei Man Ryu, Anyang-si (KR); Jin Soo Lee, Yongin-si (KR); Young Goo Jin, Gunpo-si (KR); Jae Hoon Park, Seoul (KR); Bo Kyung Kim, Anyang-si (KR); Dae Yeon Won, Anyang-si (KR); Yun-Ha Hwang, Gunpo-si (KR); Ki Yoon Kim, Hwanseong-si (KR); Sae Kwang Ku, Suwon-si (KR)

Correspondence Address: ARENT FOX LLP, 1050 CONNECTICUT AVENUE, N.W., SUITE 400, WASHINGTON, DC, 20036, US

(73) Assignee: DONG WHA PHARMACEUTICAL CO. LTD., Seoul (KR)

(21) Appl. No.: 12/670,814

(22) PCT Filed: Jul. 28, 2008
(86) PCT No.: PCT/KR08/04394

§ 371 Date: Mar. 19, 2010

(30) Foreign Application Priority Data
Jul. 27, 2007 (KR) 10-2007-0075976

(51) Int. Cl. C07D 417/02; C07D 413/02

(52) U.S. Cl. 544/60; 544/133; 544/369

(57) ABSTRACT

The present invention relates to novel benzamidine derivatives, a process for the preparation thereof and a pharmaceutical composition for preventing or treating osteoporosis comprising the same. The benzamidine derivatives of the present invention effectively inhibit osteoclast differentiation at an extremely low concentration, and thus can be advantageously used for the prevention and treatment of osteoporosis.

### TECHNICAL FIELD

The present invention relates to novel benzamidine derivatives, a process for the preparation thereof and a pharmaceutical composition for preventing or treating osteoporosis comprising the same.

### BACKGROUND ART

Bone is a supporting material for the body's framework and serves to conserve the necessary bone mass and structure. Bone also functions as a reservoir of calcium (Ca²⁺) or the like, and plays an important role in maintaining the calcium level in the blood. To this end, the growth of bone is a metabolic balance between the activity of osteoblasts and osteoclasts in the bone remodeling cycle. Accordingly, bone is in a steady state, which maintains good balance between bone absorption and bone formation in the metabolism by continuously performing both bone absorption and bone formation. When the balance between bone absorption and bone formation is disrupted, the degree of bone absorption is relatively higher than that of bone formation, which may lead to osteoporosis, a condition which causes reduction in bone density or bone mass, resulting in decrease in bone strength. This is a disease which frequently occurs in middle-aged or elderly women.

Osteoporosis is a disease which results from a disturbance in the balance between bone absorption and bone formation, and is caused by having a higher degree of bone absorption relative to that of bone formation. Osteoporosis reduces calcification of bone tissues, and decreases the level of the compact substances in the bone, which broadens the marrow cavity. As osteoporosis progresses, bone becomes brittle, and bone fracture may easily occur even with a small impact. Bone is a steady state structure, in which the bone formation by osteoblast and the bone resorption by osteoclast occur continuously.

Previous studies on osteoporosis have focused mainly on the metabolism of bone minerals, such as calcium and phosphorus. However, such studies did not provide sufficient findings on the mechanisms of osteoporosis.

Although bisphosphonate (alendronate, etidronate), hormones (raloxifen), vitamin D, calcitonin, calcium agents, or the like have been used as an anti-osteoporotic agent, they are known to have adverse effects. Specifically, bisphosphonate agents show low absorptivity and may induce esophagitis, in addition to being difficult to dose. Hormone agents must be administered throughout patient's life, and in the case of long-term administration, side effects such as breast cancer, uterine cancer, gallstones and thrombosis may be induced. Vitamin D agents are expensive and show little efficacy, while calcitonin agents are also very expensive and difficult to administer. Calcium agents have few side effects, but their effects are restricted to the prevention of osteoporosis, not the treatment itself.

It is known that osteoporosis cannot be treated with a short-term administration of drugs, and generally requires long-term administration. Therefore, there is a need for a novel substance having excellent efficacy, without the above-mentioned side effects in the long-term administration.

### DISCLOSURE

### Technical Problem

Accordingly, the present inventors have conducted extensive studies on an effective agent for treating osteoporosis, and synthesized novel benzamidine derivatives. They found that the compounds have excellent effect of inhibiting bone resorption by osteoclast and thus of treating and preventing osteoporosis, thereby completing the present invention.

### Technical Solution

It is an object of the present invention to provide novel benzamidine derivatives.

It is another object of the present invention to provide a process for the preparation of the novel benzamidine derivatives.

It is still another object of the present invention to provide a pharmaceutical composition for preventing or treating osteoporosis, comprising the novel benzamidine derivatives.

### BEST MODE

In accordance with an aspect, the present invention provides a novel benzamidine derivative represented by the following Formula 1.

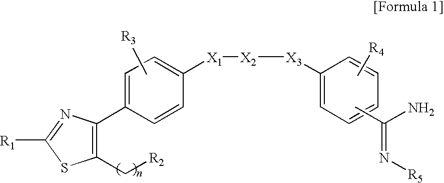

wherein,

R₁ is C₁˜C₆ alkyl which is unsubstituted or substituted with one group selected from pyridine and

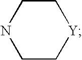

C₃˜C₆ cycloalkyl; phenyl; benzyl; pyridinyl which is unsubstituted or substituted with C₁˜C₆ alkyl; guanidino; NR₆R₇; CH₂NR₆R₇;

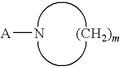

(wherein A is C₁˜C₆ alkyl, and m is an integer of 2 to 6); or

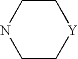

group which is unsubstituted or substituted with C₁˜C₆ alkyl;

R₂ is a primary or secondary amine, which is NR₈R₉,

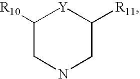

pyrrolidine, piperidine, triazole, tetrazole or imidazole;

R₃ and R₄ are each independently hydrogen; halogen; hydroxy; C₁˜C₆ alkyl which is unsubstituted or substituted with halogen; C₃˜C₆ cycloalkylamino; C₁˜C₆ alkoxy; C₁˜C₆ alkanoyloxy; C₂˜C₆ alkenyloxy; phenyl-C₁˜C₆ alkoxy; phenoxy; C₂˜C₆ alkenyloxy or phenyl-C₁˜C₆ alkanoyloxy; or C₃˜C₆ cycloalkyloxy which is substituted with one group selected from carboxy, esterified carboxy and amidated carboxy; or an aminooxy group;

R₅ is a hydrogen or hydroxy group;

R₆ and R₇ are each independently hydrogen; C₁˜C₆ alkyl which is unsubstituted or substituted with one group selected from hydroxy, C₁˜C₆ alkoxy, pyridine and

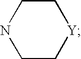

phenyl; benzyl; pyridinyl; carbonyl which is substituted with one group selected from C₁˜C₆ alkyl, hydroxy, C₁˜C₆ alkoxy, phenyl, benzyl, pyridine and

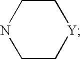

or C₁˜C₆ alkanesulfonyl;

R₈ and R₉ are each independently hydrogen; C₁˜C₆ alkyl which is unsubstituted or substituted with one group selected from hydroxy, C₁˜C₆ alkoxy, morpholine, imidazole and NR₆R₇; C₁˜C₆ alkoxy; C₃˜C₆ cycloalkyl; phenyl; benzyl; pyridinyl; morpholine; carbonyl which is substituted with one group selected from C₁˜C₆ alkyl, C₁˜C₆ alkoxy, phenyl, benzyl, pyridine and

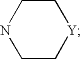

carbonyl substituted with C₁˜C₆ alkyl which is substituted with one group selected from halogen, C₁˜C₆ alkoxy and imidazole; or C₁˜C₆ alkanesulfonyl;

R₁₀ and R₁₁ are each independently hydrogen, C₁˜C₂ alkyl, C₁˜C₃ alkoxy or halide;

X₁ and X₃ are each independently O; S; NH; or N—C₁˜C₆ alkyl, N—C₃˜C₆ cycloalkyl, N-benzyl or N-phenyl group;

X₂ is C₃˜C₇ alkylene; C₁˜C₃ alkylene-C₂-C₇ alkenylene-C₁˜C₃ alkylene; C₁˜C₃ alkylene-O—C₁˜C₃ alkylene; C₁˜C₃ alkylene-S—C₁˜C₃ alkylene; C₁˜C₃ alkylene-NH—C₁˜C₃ alkylene; C₁˜C₃ alkylene-pyridylene-C₁˜C₃ alkylene or C₁˜C₃ alkylene-naphthylene-C₁˜C₃ alkylene; C₃˜C₇ alkylene which is substituted with C₁˜C₃ alkyl and hydroxyl; C₃˜C₇ alkylene carbonyl; or C₃˜C₇ alkylene which is interrupted by piperazine;

Y is O, S, NR₆ or CH₂ group; and

N is an integer of 0 or 1.

In Formula 1, R₁ is particularly methyl, ethyl, isopropyl, phenyl, pyridinyl, cyclohexyl, morpholinyl,

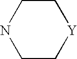

which is unsubstituted or substituted with C₁˜C₆ alkyl, NR₆R₇ or CH₂NR₆R₇;

R₂ is a primary or secondary amine, which is NR₈R₉,

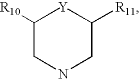

piperidine, pyrrolidine, imidazole or triazole;

R₃ and R₄ are each independently hydrogen, methyl, ethyl, halogen, hydroxy or methoxy group;

R₅ is a hydroxy group;

R₆ and R₇ are each independently hydrogen, methyl, ethyl, propyl, hydroxyethyl, methoxyethyl, 2-morpholinoethyl, benzyl, pyridin-3-ylmethyl, pyridin-4-ylmethyl, 3-pyridinylcarbonyl or ethanesulfonyl;

R₈ and R₉ are each independently hydrogen; methyl; ethyl; propyl; isopropyl; butyl; isobutyl; t-butyl; hydroxyethyl; methoxyethyl; 2-morpholinoethyl; benzyl; 3-imidazole-lyl-propyl; cyclopropyl; or carbonyl which is substituted with one group selected from 3-pyridinyl and 4-pyridinyl;

R₁₀ and R₁₁ are each independently hydrogen or methyl;

X₁ and X₃ are each independently oxygen, sulfur, amine or methylamine group;

X₂ is propylene, butylene, pentylene, hexylene, ethylene-O-ethylene, ethylene-NH-ethylene, butylene carbonyl, 2-butenyl, methylene-1,2-phenylene-methylene, methylene-1,3-phenylene-methylene, methylene-1,4-phenylene-methylene or methylene-pyridinyl-methylene;

Y is O, S or methylamino or CH₂ group; and

n is an integer of 0 or 1.

In the compound of Formula 1 of the present invention, R₃ and R₄ are in the ortho or meta position relative to —O— (CH₂)₅—O—, and —C(NH₂)═N—R₅ is in the meta or para position.

The preferred compounds among the benzamidine derivatives of Formula 1 of the present invention are as follows:
1) N-hydroxy-4-{5-[4-(2-methyl-5-morpholin-4-yl-thiazol-4-yl)-phenoxy]-pentyloxy}-benzamidine,
2) N-hydroxy-4-{5-[(4-[2-methyl-5-(4-methyl-piperazin-1-yl)-thiazol-4-yl]-phenoxy}-pentyloxy)-benzamidine,
3) N-hydroxy-4-{5-[4-(2-amino-5-morpholin-4-yl-thiazol-4-yl)-phenoxy]-pentyloxy}-benzamidine,
4) N-hydroxy-4-(5-{4-[5-(4-methyl-piperazin-1-yl)-2-morpholin-4-yl-thiazol-4-yl]-phenoxy}-pentyloxy)-benzamidine,
5) N-hydroxy-4-{5-[4-(2,5-di-morpholin-4-yl-thiazol-4-yl)-phenoxy]-pentyloxy}-benzamidine,
6) N-hydroxy-4-{5-[(4-(2-morpholin-4-yl-5-thiomorpholin-4-yl-thiazol-4-yl)-phenoxy]-pentyloxy}-benzamidine,
7) N-hydroxy-4-{5-[4-(2-morpholin-4-yl-5-pyrrolidin-1-yl-thiazol-4-yl)-phenoxy]-pentyloxy}-benzamidine,
8) N-hydroxy-4-{5-[4-(2-methyl-5-morpholin-4-ylmethyl-thiazol-4-yl)-phenoxy]-pentyloxy}-benzamidine,
9) N-hydroxy-4-(5-{4-[2-methyl-5-(4-methyl-piperazin-1-ylmethyl)-thiazol-4-yl]-phenoxy}-pentyloxy)-benzamidine,
10) N-hydroxy-4-{5-[4-(2-methyl-5-thiomorpholin-4-ylmethyl-thiazol-4-yl)-phenoxy]-pentyloxy}-benzamidine,
11) N-hydroxy-4-{5-[4-(2-methyl-5-piperidin-1-ylmethyl-thiazol-4-yl)-phenoxy]-pentyloxy}-benzamidine,
12) N-hydroxy-4-{5-[4-(5-dimethylaminomethyl-2-methyl-thiazol-4-yl)-phenoxy]-pentyloxy}-benzamidine,
13) N-hydroxy-4-{5-[4-(5-butylaminomethyl-2-methyl-thiazol-4-yl)-phenoxy]-pentyloxy}-benzamidine,
14) N-hydroxy-4-(5-{4-[5-(isobutylamino-methyl)-2-methyl-thiazol-4-yl]-phenoxy}-pentyloxy)-benzamidine,
15) N-hydroxy-4-(5-{4-[5-(tert-butylamino-methyl)-2-methyl-thiazol-4-yl]-phenoxy}-pentyloxy)-benzamidine,
16) N-hydroxy-4-{5-[4-(2-methyl-5-propylaminomethyl-thiazol-4-yl)-phenoxy]-pentyloxy}-benzamidine,
17) N-hydroxy-4-[5-(4-{2-methyl-5-[(2-morpholin-4-yl-ethylamino)-methyl]-thiazol-4-yl}-phenoxy)-pentyloxy]-benzamidine,
18) M-hydroxy-4-[5-(4-{5-[(3-imidazol-1-yl-propylamino)-methyl]-2-methyl-thiazol-4-yl}-phenoxy)-pentyloxy]-benzamidine,
19) N-hydroxy-4-{5-[4-(2-methyl-5-pyrrolidin-1-ylmethyl-thiazol-4-yl)-phenoxy]-pentyloxy}-benzamidine,
20) N-hydroxy-4-{5-[4-(5-imidazol-1-ylmethyl-2-methyl-thiazol-4-yl)-phenoxy]-pentyloxy}-benzamidine,
21) N-hydroxy-4-(5-{4-[5-(benzylamino-methyl)-2-methyl-thiazol-4-yl]-phenoxy}-pentyloxy)-benzamidine,
22) N-hydroxy-4-{5-[4-(5-cyclopropylaminomethyl-2-methyl-thiazol-4-yl)-phenoxy]-pentyloxy}-benzamidine,
23) N-hydroxy-4-{5-[4-(2-methylamino-5-morpholin-4-yl-thiazol-4-yl)-phenoxy]-pentyloxy}-benzamidine,
24) N-hydroxy-4-(5-{4-[2-(methyl-pyridin-4-ylmethyl-amino)-5-morpholin-4-ylmethyl-thiazol-4-yl]-phenoxy}-pentyloxy)-benzamidine,
25) N-hydroxy-4-[5-(4-{2-[(2-hydroxy-ethyl)-methyl-amino]-5-morpholin-4-ylmethyl-thiazol-4-yl}-phenoxy)-pentyloxy]-benzamidine,
26) N-hydroxy-4-(5-{4-[2-(ethyl-methyl-amino)-5-morpholin-4-ylmethyl-thiazol-4-yl]-phenoxy}-pentyloxy)-benzamidine,
27) N-hydroxy-4-(5-{4-[2-(benzyl-methyl-amino)-5-morpholin-4-yl methyl-thiazol-4-yl]-phenoxy}-pentyloxy)-benzamidine,
28) N-hydroxy-4-[5-(4-{2-[methyl-(2-morpholin-4-yl-ethyl)-amino]-5-morpholin-4-ylmethyl-thiazol-4-yl}-phenoxy)-pentyloxy]-benzamidine,
29) N-hydroxy-4-[5-(4-{2-[methyl-(2-morpholin-4-yl-ethyl)-amino]-5-thiomorpholin-4-ylmethyl-thiazol-4-yl}-phenoxy)-pentyloxy]-benzamidine,
30) N-hydroxy-4-[5-(4-{5-{[bis-(2-methoxy-ethyl)-amino]-methyl}-2-[methyl-(2-morpholin-4-yl-ethyl)-amino]-thiazol-4-yl}-phenoxy)-pentyloxy]-benzamidine,
31) N-hydroxy-4-(5-{4-[2-[methyl-(2-morpholin-4-yl-ethyl)-amino]-5-(4-methyl-piperazin-1-ylmethyl)-thiazol-4-yl]-phenoxy}-pentyloxy)-benzamidine,
32) N-hydroxy-4-[5-(4-{5-(isopropylamino-methyl)-2-[methyl-(2-morpholin-4-yl-ethyl)-amino]-thiazol-4-yl}-phenoxy)-pentyloxy]-benzamidine,
33) N-hydroxy-4-[5-(4-{5-[(2-methoxy-ethylamino)-methyl]-2-[methyl-(2-morpholin-4-yl-ethyl)-amino]-thiazol-4-yl}-phenoxy)-pentyloxy]-benzamidine,
34) N-hydroxy-4-[5-(4-{2-[(2-methoxy-ethyl)-methyl-amino]-5-morpholin-4-ylmethyl-thiazol-4-yl}-phenoxy)-pentyloxy]-benzamidine,
35) N-hydroxy-4-(5-{4-[2-(methyl-propyl-amino)-5-morpholin-4-yl methyl-thiazol-4-yl]-phenoxy}-pentyloxy)-benzamidine,
36) N-hydroxy-4-(5-{4-[2-(methyl-pyridin-3 ylmethyl-amino)-5-morpholin-4-ylmethyl-thiazol-4-yl]-phenoxy}-pentyloxy)-benzamidine,
37) N-hydroxy-4-{5-[4-(2-methyl-5-methylamino-thiazol-4-yl)-phenoxy]-pentyloxy}-benzamidine,
38) N-hydroxy-4-[5-(4-{2-methyl-5-[(pyridine-4-carbonyl)-amino]-thiazol-4-yl}-phenoxy)-pentyloxy]-benzamidine,
39) N-hydroxy-4-[5-(4-{2-methyl-5-[(pyridine-3-carbonyl)-amino]-thiazol-4-yl}-phenoxy)-pentyloxy]-benzamidine,
40) N-hydroxy-4-[5-(4-{2-phenyl-5-[(pyridine-3-carbonyl)-amino]-thiazol-4-yl}-phenoxy)-pentyloxy]-benzamidine,
41) N-hydroxy-4-{5-[4-(5-dimethylamino-2-methyl-thiazol-4-yl)-phenoxy-pentyloxy}-benzamidine,
42) N-hydroxy-4-{5-[4-(5-dimethylamino-2-phenyl-thiazol-4-yl)-phenoxy]-pentyloxy}-benzamidine,
43) N-hydroxy-4-{5-[4-(2-cyclohexyl-5-dimethylamino-thiazol-4-yl)-phenoxy]-pentyloxy}-benzamidine,
44) N-hydroxy-4-{5-[(4-(2-methyl-5-[1,2,4]triazol-1-yl-thiazol-4-yl)-phenoxy]-pentyloxy}-benzamidine,
45) N-hydroxy-4-{5-[4-(5-amino-2-phenyl-thiazol-4-yl)-phenoxy]-pentyloxy}-benzamidine,
46) N-hydroxy-4-{5-[4-(5-amino-2-methyl-thiazol-4-yl)-phenoxy]-pentyloxy}-benzamidine,
47) N-hydroxy-4-{5-[4-(5-amino-2-pyridin-3-yl-thiazol-4-yl)-phenoxy]-pentyloxy}-benzamidine,
48) N-hydroxy-4-{5-[4-(5-amino-2-ethyl-thiazol-4-yl)-phenoxy]-pentyloxy}-benzamidine,
49) N-hydroxy-4-{5-[4-(5-amino-2-cyclohexyl-thiazol-4-yl)-phenoxy]-pentyloxy}-benzamidine,
50) N-hydroxy-4-{5-[4-(2-methylamino-5-morpholin-4-ylmethyl-thiazol-4-yl)-phenoxy]-pentyloxy}-benzamidine,
51) N-hydroxy-4-{5-[4-(2-morpholin-4-yl-5-morpholin-4-ylmethyl-thiazol-4-yl)-phenoxy]-pentyloxy}-benzamidine,
52) N-hydroxy-4-{5-[4-(5-morpholin-4-yl-2-piperidin-1-yl-thiazol-4-yl)-phenoxy]-pentyloxy}-benzamidine.

The benzamidine derivatives of the formula 1 of the present invention may be used in the form of pharmaceutically acceptable salts. Preferable are acid addition salts prepared with pharmaceutically acceptable free acids. Free acids suitable for use in the present invention may be inorganic acids or organic acids. Examples of the inorganic acids may include hydrochloric acid, bromic acid, sulfuric acid, phosphoric acid, and the organic acids may be exemplified by citric acid, acetic acid, lactic acid, tartaric acid, fumaric acid, formic acid, propionic acid, oxalic acid, trifluoroacetic acid, methane sulfonic acid, benzene sulfonic acid, maleic acid, benzoic acid, gluconic acid, glycolic acid, succinic acid, 4-morpholine ethane sulfonic acid, camphorsulfonic acid, 4-nitrobenzene sulfonic acid, hydroxy-O-sulfonic acid, 4-toluene sulfonic acid, galacturonic acid, embonic acid, glutamic acid, aspartic acid. Preferably, hydrochloric acid as inorganic acid and methane sulfonic acid as organic acid can be used.

In the present invention, general definitions of the substituents of the compound of Formula 1 have the following meanings:

The term “halogen” means halogen group atoms including chlorine, fluorine, bromine, and iodine radicals.

The term “alkyl” means straight or branched, saturated hydrocarbon radicals having 1 to 6 carbon atoms, and examples thereof include methyl, ethyl, n-propyl, iso-propyl, n-butyl, sec-butyl and tert-butyl.

The term “alkoxy” means radicals having straight or branched alkyl having 1 to 6 carbon atoms that is linked to oxygen, and examples thereof include methoxy, ethoxy, propoxy, iso-propoxy, butoxy, sec-butoxy, and tert-butoxy.

The term “cycloalkyl” means a non-aromatic hydrocarbon ring having 3 to 6 carbon atoms, and examples thereof include cyclopropyl, cyclobutyl, cyclopentyl and cyclohexyl.

The term “alkenyl” means straight or branched, unsaturated hydrocarbons having 2 to 6 carbon atoms with one or more double bonds.

The term “alkanoyloxy” means an oxygen-containing radical in which a terminal carbon atom of an alkyl group is substituted with a carbonyl radical.

The term “alkanoyloxy” means an oxygen-containing radical in which a terminal carbon atom of an alkenyl group is substituted with a carbonyl radical.

The term “alkenyloxy” means an oxygen-containing alkenyl group.

The term “alkylene” means a straight or branched, saturated hydrocarbon radical having 1 to 7 carbon atoms, and 2 or more junction centers for a covalent bond, and examples thereof include methylene, ethylene, methylethylene and isopropylidene.

The term “alkenylene” means a straight or branched, unsaturated hydrocarbon radical having 2 to 7 carbon atoms, 2 or more conjunction centers for a covalent bond and one or more double bonds, and examples thereof include-1,1-vinylidene (CH₂═C), 1,2-vinylidene (—CH═CH—), and 1,4-butadienyl (—CH═CH—CH═CH—).

The term “carbonyl” means a carbon radical in which 2 of 4 covalent bonds are linked to oxygen atoms.

In accordance with another aspect, the present invention provides a process for the preparation of the benzamidine derivative of Formula 1.

The compound of Formula 1, wherein R₁ is methyl, ethyl, isopropyl, phenyl, morpholinyl or amino, can be prepared as in the following Reaction Scheme 1 comprising the steps of 1) to 7):

1) reacting a compound of Formula 2 with a compound of Formula 3 in the presence of an inorganic base to prepare a compound of Formula 4,

2) reacting a compound of Formula 5 with the compound of Formula 4 obtained in step 1) in the presence of an inorganic base to prepare a compound of Formula 6,

3) reacting the compound of Formula 6 obtained in step 2) with a bromine compound to prepare a benzonitrile derivative of Formula 7,

4) reacting the alpha-brominated compound of Formula 7 obtained in step 3) with a thioamide compound of Formula 8 to prepare a benzonitrile derivative having a thiazole group of Formula 9,

5) reacting the compound of Formula 9 obtained in step 4) with a bromine compound to prepare a benzonitrile derivative having a brominated thiazole group of Formula 10,

6) reacting the compound of Formula 10 obtained in step 5) with a primary or secondary amine compound of Formula 11 to prepare a benzonitrile derivative of Formula 12, and

7) reacting the compound of Formula 12 obtained in step 6) with a hydroxylamine or hydrochloric alcohol solution and ammonia to prepare a benzamidine derivative of Formula 1a.

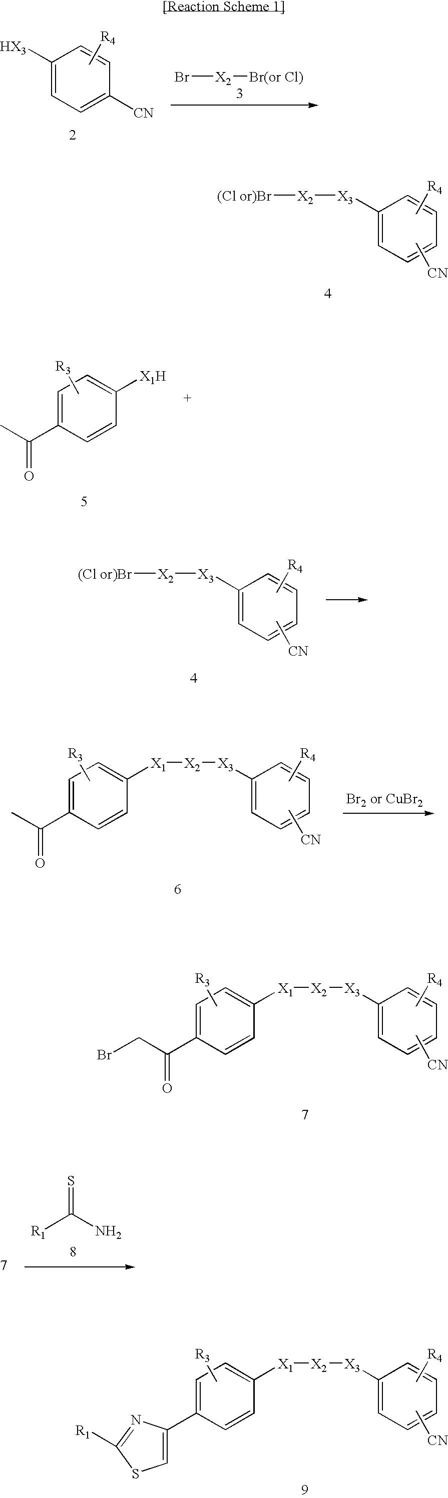

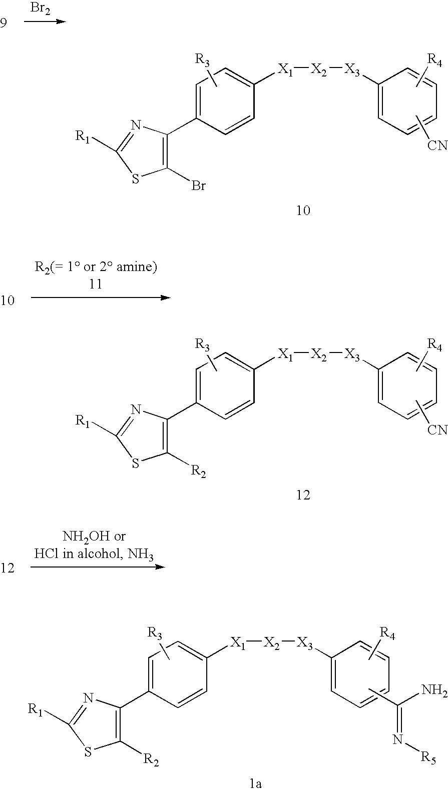

wherein. R₁ is methyl, ethyl, isopropyl, phenyl, morpholinyl or amino, and R₂, R₃, R₄, R₅, X₁, X₂ and X₃ are the same as defined in the compound of Formula 1.

The compound of Formula 1, wherein R₁ is methyl, ethyl, isopropyl, or phenyl and n is 1, can be prepared as in the following Reaction Scheme 2 comprising the steps of 1) to 6):

1) reacting the compound of Formula 4 obtained in step 1) of Reaction Scheme 1 with a compound of Formula 13 to prepare a benzonitrile derivative of Formula 14,

2) reacting the compound of Formula 14 obtained in step 1) with a bromine compound to prepare an alpha-brominated benzonitrile derivative of Formula 15,

3) reacting the alpha-brominated compound of Formula 15 obtained in step 2) with a thioamide compound of Formula 8 to prepare a benzonitrile derivative having a thiazole group of Formula 16,

4) reacting the compound of Formula 16 obtained in step 3) with a bromine compound to prepare a benzonitrile derivative having a brominated thiazole group of Formula 17,

5) reacting the compound of Formula 17 obtained in step 4) with the primary or secondary amine compound of Formula 11 to prepare a benzonitrile derivative of Formula 18, and

6) reacting the compound of Formula 18 obtained in step 5) with a hydroxylamine or hydrochloric alcohol solution and ammonia to prepare a benzamidine derivative of Formula 1b.

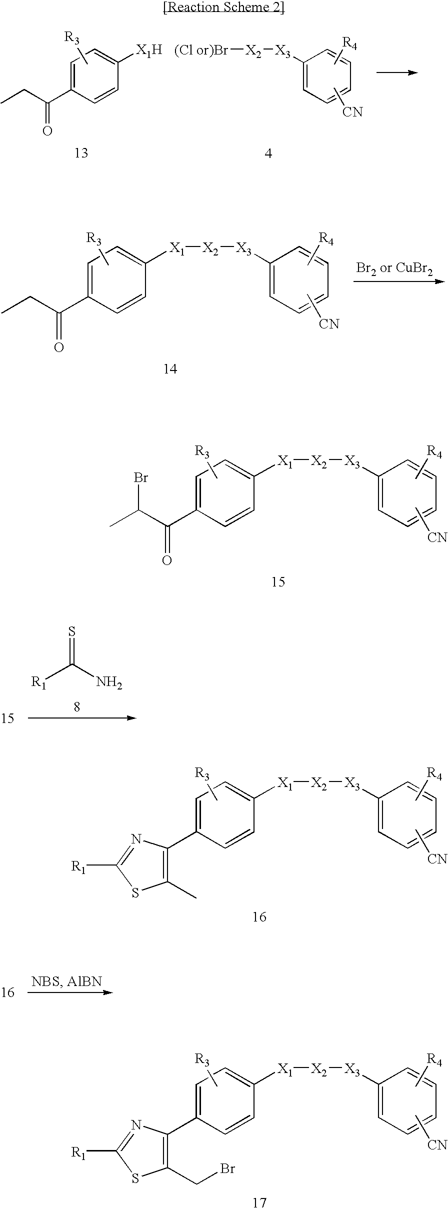

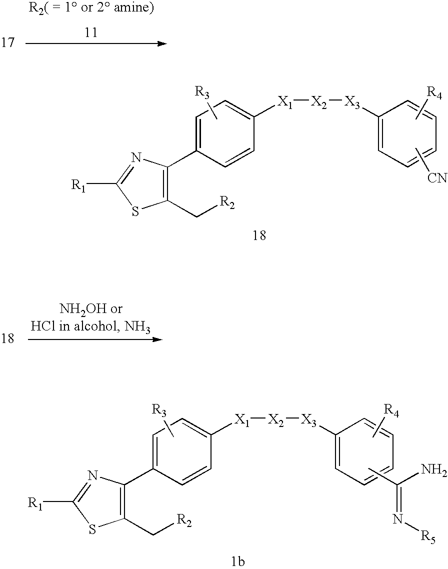

wherein R₁ is methyl, ethyl, isopropyl, or phenyl, and R₂, R₃, R₄, R₅, X₁, X₂ and X₃ are the same as defined in the compound of Formula 1.

The compound of Formula 1, wherein R₁ is CH₂NHR₆ or NHR₆ (except that R₆ is hydrogen) and n is 1, can be prepared as in the following Reaction Scheme 3 comprising the steps of 1) to 4):

1) reacting the compound of Formula 7 obtained in step 3) of Reaction Scheme 1 with a thiourea compound (19) to prepare a benzonitrile derivative having an amino-thiazole group of Formula 20,

2) reacting the compound of Formula 20 obtained in step 1) with a bromine compound to prepare a benzonitrile derivative having a brominated amino-thiazole group of Formula 21,

3) reacting the compound of Formula 21 obtained in step 2) with the primary or secondary amine compound of Formula 11 to prepare a benzonitrile derivative of Formula 22, and

4) reacting the compound of Formula 22 obtained in step 3) with a hydroxylamine or hydrochloric alcohol solution and ammonia to prepare a benzamidine derivative of Formula 1c.

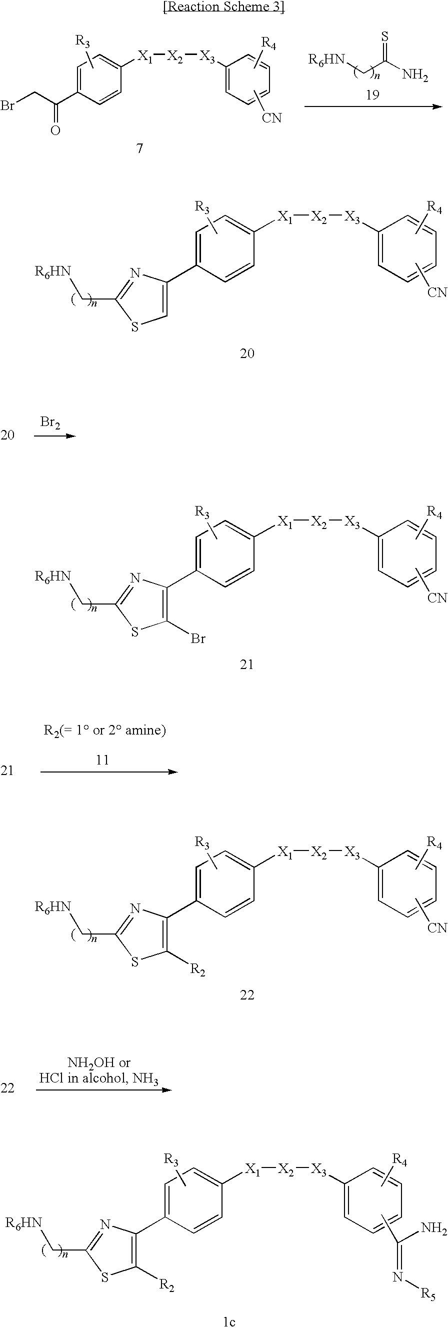

wherein R₂, R₃, R₄, R₅, R₆, X₁, X₂ and X₃ are the same as defined in the compound of Formula 1.

The compound of Formula 1, wherein R₁ is CH₂NR₆R₇ or NR₆R₇ (except that both R₆ and R₇ are hydrogen), can be prepared as in the following Reaction Scheme 4 comprising the steps of 1) to 3):

1) reacting the compound of Formula 20 obtained in step 1) of Reaction Scheme 3 with a compound of Formula 23 to prepare a benzonitrile derivative having a thiazole group of Formula 24,

2) reacting the compound of Formula 24 obtained in step 1) with formaldehyde and the primary or secondary amine compound of Formula 11 to prepare a benzonitrile derivative of Formula 25, and

3) reacting the compound of Formula 25 obtained in step 2) with a hydroxylamine or hydrochloric alcohol solution and ammonia to prepare a benzamidine derivative of Formula 1d.

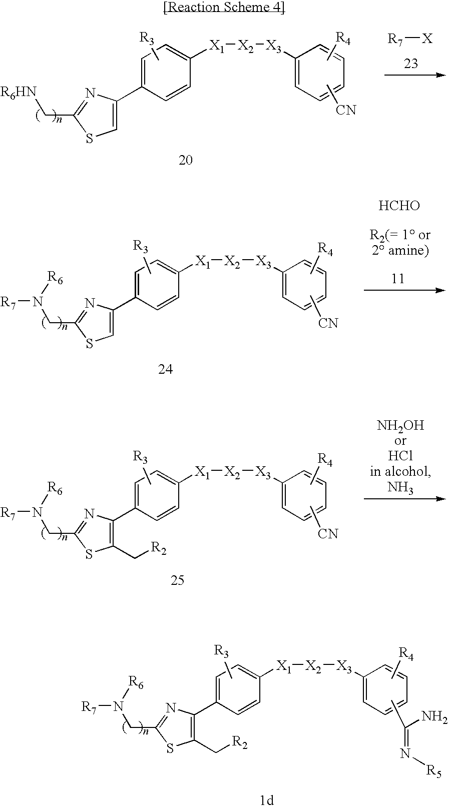

wherein R₂, R₃, R₄, R₅, R₆, R₇, X₁, X₂, X₃ and n are the same as defined in the compound of Formula 1.

The compound of Formula 1, wherein R₁ is methyl, ethyl, isopropyl, phenyl, pyridinyl, or cyclohexyl, can be prepared as in the following Reaction Scheme 5 comprising the steps of 1) to 4):

1) reacting the compound of Formula 9 obtained in step 4) of Reaction Scheme 1 with nitric acid to prepare a benzonitrile derivative having a thiazole group containing a nitrous acid group of Formula 26,

2) reacting the compound of Formula 26 obtained in step 1) with iron or tin chloride dihydrate to prepare a benzonitrile derivative having an amino-thiazole group of Formula 27,

3) reacting the compound of Formula 27 obtained in step 2) with a halide compound of Formula 28 to prepare a benzonitrile derivative substituted with a primary amine of Formula 29, and

4) reacting the compound of Formula 29 obtained in step 3) with a hydroxylamine or hydrochloric alcohol solution and ammonia to prepare a benzamidine derivative of Formula 1e.

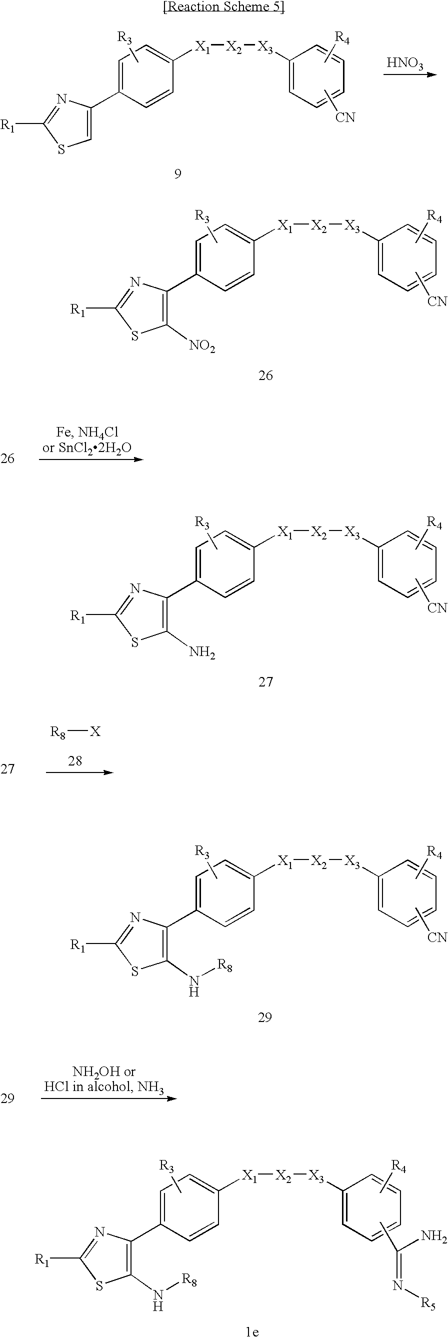

wherein R₁ is methyl, ethyl, isopropyl, phenyl, pyridinyl, or cyclohexyl, and R₃, R₄, R₅, R₈, X₁, X₂ and X₃ are the same as defined in the compound of Formula 1.

The compound of Formula 1, wherein R₁ is methyl, ethyl, isopropyl, phenyl, pyridinyl, or cyclohexyl, can be prepared as in the following Reaction Scheme 6 comprising the steps of 1) and 2):

1) reacting the compound of Formula 27 obtained in step 2) of Reaction Scheme 5 with a halide compound of Formula 28 to prepare a benzonitrile derivative substituted with a primary amine of Formula 30, and

2) reacting the compound of Formula 30 obtained in step 1) with a hydroxylamine or hydrochloric alcohol solution and ammonia to prepare a benzamidine derivative of Formula 1f.

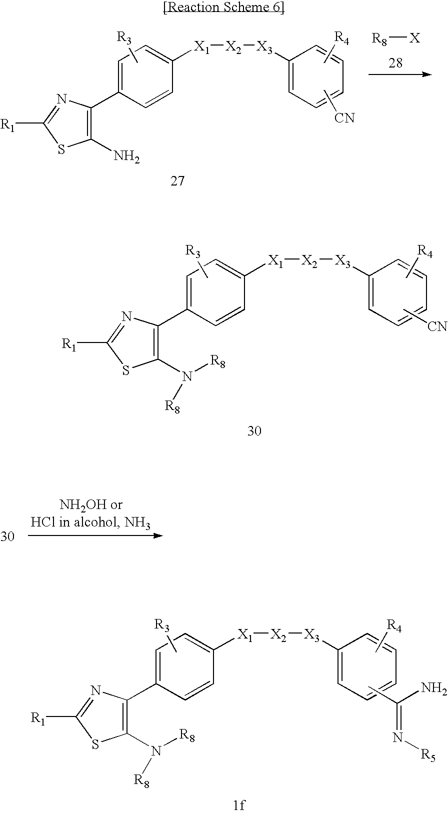

wherein R₁ is methyl, ethyl, isopropyl, phenyl, pyridinyl, or cyclohexyl, and R₃, R₄, R₅, R₈ (except that R₈ is hydrogen), X₁, X₂ and X₃ are the same as defined in the compound of Formula 1.

The compound of Formula 1, wherein R₁ is methyl, ethyl, isopropyl, or phenyl, can be prepared as in the following Reaction Scheme 7 comprising the steps of 1) and 4):

1) reacting the compound of Formula 7 obtained in step 3) of Reaction Scheme 1 with the primary or secondary amine of Formula 11 to prepare a benzonitrile derivative of Formula 31,

2) reacting the compound of Formula 31 obtained in step 1) with a bromine compound to prepare an alpha-brominated compound of Formula 32,

3) reacting the compound of Formula 32 obtained in step 2) with the thioamide compound of Formula 8 to prepare a benzonitrile derivative having a thiazole group of Formula 12, and

4) reacting the benzonitrile derivative of Formula 12 obtained in step 3) with a hydroxylamine or hydrochloric alcohol solution and ammonia to prepare the benzamidine derivative of Formula 1a.

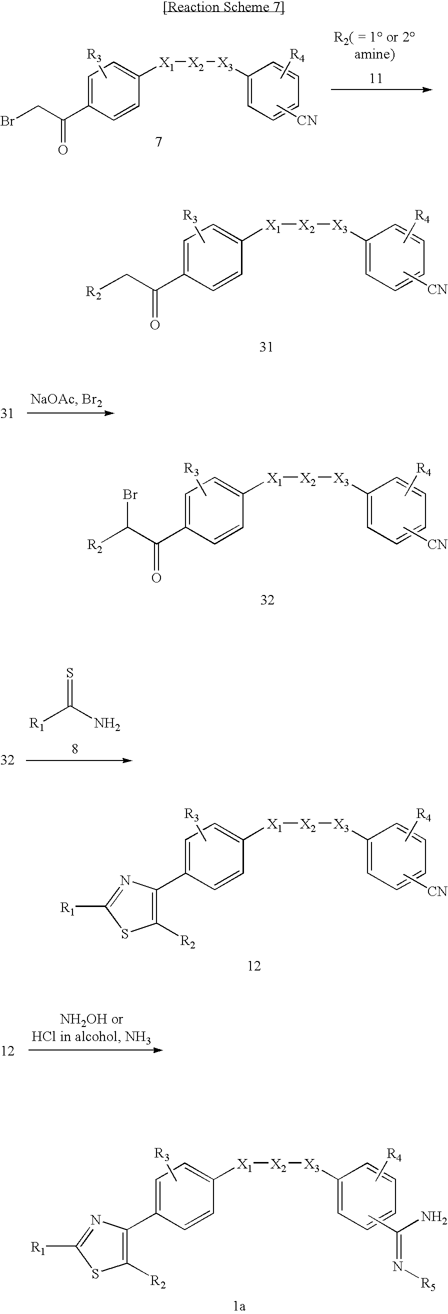

wherein R₁ is methyl, ethyl, isopropyl, or phenyl, and R₂, R₃, R₄, R₅, X₁, X₂ and X₃ are the same as defined in the compound of Formula 1.

The compound of Formula 1, wherein R₁ is methyl, ethyl, isopropyl, phenyl, pyridinyl, or cyclohexyl, can be prepared as in the following Reaction Scheme 8 comprising the step of 1):

1) reacting the compound of Formula 27 obtained in step 2) of Reaction Scheme 5 with a hydroxylamine or hydrochloric alcohol solution and ammonia to prepare a benzamidine derivative of Formula 1g.

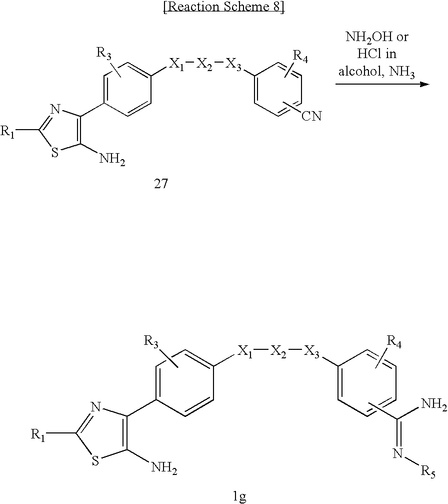

wherein R₁ is methyl, ethyl, isopropyl, phenyl, pyridinyl, or cyclohexyl, and R₃, R₅, X₁, X₂ and X₃ are the same as defined in the compound of Formula 1.

The compound of Formula 1, wherein R₁ is

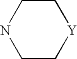

which is unsubstituted or substituted with C₁˜C₆ alkyl, CH₂NR₆R₇ or NR₆R₇ (except that both R₆ and R₇ are hydrogen), can be prepared as in the following Reaction Scheme 9 comprising the step of 1) and 2):

1) reacting the compound of Formula 9 obtained in step 4) of Reaction Scheme 1 with formaldehyde and the primary or secondary amine compound of Formula 11 to prepare the benzonitrile derivative of Formula 18, and

2) reacting the compound of Formula 18 obtained in step 1) with a hydroxylamine or hydrochloric alcohol solution and ammonia to prepare the benzamidine derivative of Formula 1b.

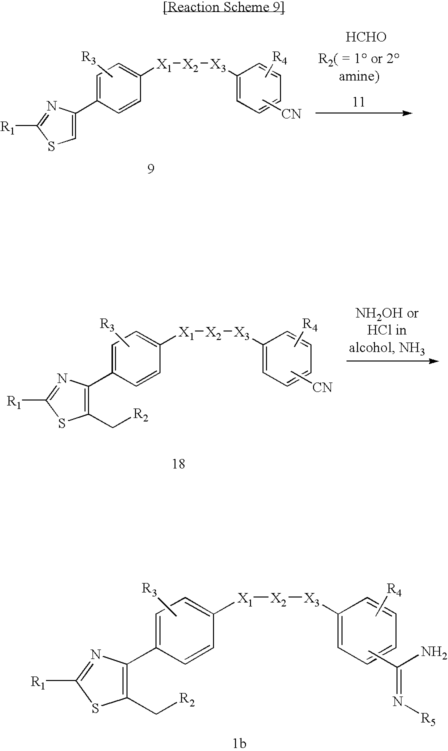

wherein R₁ is

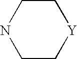

which is unsubstituted or substituted with C₁˜C₆ alkyl, CH₂NR₆R₇ or NR₆R₇ (except that both R₆ and R₇ are hydrogen), and R₂, R₃, R₄, R₅, R₆, R₇, X₁, X₂, X₃ and Y are the same as defined in the compound of Formula 1.

The compound of Formula 1, wherein R₁ is

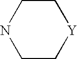

which is unsubstituted or substituted with C₁˜C₆ alkyl, can be prepared as in the following Reaction Scheme 10 comprising the step of 1) and 5):

1) reacting the compound of Formula 7 obtained in step 3) of Reaction Scheme 1 with a thiourea compound to prepare the benzonitrile derivative having an amino-thiazole group of Formula 33,

2) reacting the compound of Formula 33 obtained in step 1) with a compound of Formula 34, of which both terminals are substituted with halogen, to prepare a benzonitrile derivative of Formula 35 with a thiazole ring, which is substituted with a heteroring,

3) reacting the compound of Formula 35 obtained in step 2) with a bromine compound to prepare a benzonitrile derivative having a brominated amino-thiazole group of Formula 36,

4) reacting the compound of Formula 36 obtained in step 3) with the primary or secondary amine compound of Formula 11 to prepare a benzonitrile derivative of Formula 37, and

5) reacting the compound of Formula 37 obtained in step 4) with a hydroxylamine or hydrochloric alcohol solution and ammonia to prepare a benzamidine derivative of Formula 1h.

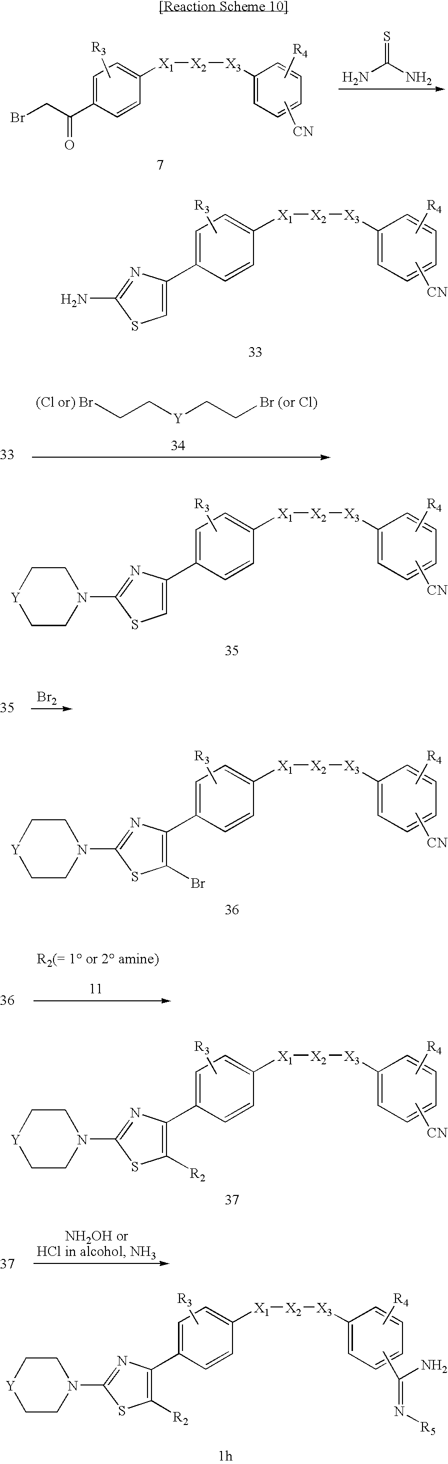

wherein R₂, R₃, R₄, R₅, X₁, X₂, X₃ and Y are the same as defined in the compound of Formula 1.

The preparation method of benzamidine derivative substituted with a thiazole derivative of the present invention is specifically described as below:

In Reaction Schemes 1 to 9, the compound (2), the compound (4), the compound (5), the compound (6), amine (11), the compound (13), the compound (14), thioamide (8), the halide compounds (23 and 28), the substituted compound (3), of which both terminals are substituted with halogen, and the compound (34) are commercially available, or can be simply synthesized for use by a method known in the art.

Reaction Scheme 1 will be described by using specific compounds.

In step 1), 4-cyanophenol (2; R₄═H, X₂═O) is reacted with 1-bromo-5-chloropentane (3; Br—X₂—Cl: X₂=pentylene) in the presence of a base to prepare 4-(5-chloropentoxy)benzonitrile (4). The base to be used herein may be an inorganic base, preferably one selected from the group consisting of potassium carbonate, sodium hydroxide, and sodium hydride. The reaction is preferably carried out at a temperature in the range of 10 to 90° C. for 1 to 9 hours, and acetonitrile, dimethylformamide, or the like is preferably used as the reaction solvent.

In step 2), 4-(5-chloropentoxy)benzonitrile derivative (4) prepared in step 1) is reacted with 4-hydroxy acetophenone (5; R₃═H, X₁═O) in the presence of a base to prepare 4-[5-(4-acetyl-phenoxy)-pentyloxy]-benzonitrile (6). The base to be used for preparing the compound (6) may be an inorganic base, and preferably one selected from the group consisting of potassium carbonate, sodium hydroxide, and sodium hydride. The reaction is preferably carried out at a temperature in the range of 10 to 90° C. for 1 to 9 hours, and acetonitrile, dimethylformamide, or the like is preferably used as the reaction solvent.

In step 3), 4-[5-(4-acetyl-phenoxy)-pentyloxy]-benzonitrile derivative (6) prepared in step 2) is reacted with a bromine compound to prepare an alpha-brominated compound, 4-{5-[4-(2-bromo-acetyl)-phenoxy]-pentyloxy}-benzonitrile (7). At this time, the reagent to be used for the reaction can be copper bromide (II) or bromine, and the reaction is preferably carried out at a temperature in the range of 20 to 80° C. for 8 to 24 hours, and ethyl acetate, dichloromethane, chloroform, or the like is used as the reaction solvent.

In step 4), the alpha-brominated compound (7) prepared in step 3) is reacted with the thioamide compound (8) to prepare a compound having a thiazole ring (9). The thioamide compound (8) to be used for the reaction is a substance to introduce the substituent R₁ into the compound of Formula 1, and the thioamide compound (8) with a proper substituent can be selected according to the type of the substituents. The reaction temperature and time may vary according to the type of the thioamide compound (8), and the reaction is preferably carried out at a temperature in the range of 60 to 90° C. for 5 to 24 hours. Examples of the thioamide compound (8) include thioacetamide, thiopropionamide, thioisobutyramide, trimethylthioacetamide, thiohexanoamide, cyclohexancarbothioicacidamide, N-(2-amino-2-thioxoethyl)-2-methylpropanamide, piperidine-4-carbothioicacidamide, morpholin-4-carbothioicacidamide, thiourea, amidino thiourea, thiobenzamide, glycine thioamide, 2,2-dimethylthiopropionamide, N-methylthiourea, N-ethylthiourea, and N-propylthiourea, which are available commercially or simply synthesized by a method known in the art. In addition, a single solvent of ethanol or a mixed solvent of ethanol and water is used as the reaction solvent.

In step 5), the compound (9) having a thiazole ring prepared in step 4) is reacted with bromine to prepare a compound (10). The reaction is preferably carried out at a temperature in the range of 0 to 80° C. for 1 to 4 hours, and chloroform, dichloromethane, or ethyl acetate is preferably used as the reaction solvent.

In step 6), the compound (10) prepared in step 5) is reacted with a primary or secondary amine compound (11) to prepare a compound (12). The amide compound (11) to be used for preparing the compound (12) is a substance to introduce the substituent R₂ into the compound of Formula 1 and the amine compound (11) with a proper substituent can be selected according to the type of the substituents. Examples of the amine compound (11) include methylamine, dimethylamine, ethylamine, diethylamine, propylamine, isopropylamine, diisopropylamine, butylamine, dibutylamine, t-butylamine, isopropyloxypropylamine, piperidine, pyrrolidine, morpholine, pyrimidine, imidazole, N-methylpiperazine, N-methylethylamine, N,N-dimethylethylamine, dimethoxyethylamine, isobutyrylamine, dihydroxyethylamine, 2,6-dimethylmorpholine, thiomorpholine, aminoethylmorpholine, aminopropylimidazole, aminopropylmorpholine, aminoethylimidazole, cyclopentylamine, cyclopropylamine, and cyclohexylamine, which are available commercially or simply synthesized by a method known in the art. The reaction is preferably carried out at a temperature in the range of 20 to 180° C. for 1 to 24 hours. Further, acetonitrile, dimethylformamide, or the like is preferably used as the reaction solvent, or the amine compound may be singly used without any solvent.

In step 7, the compound (12) prepared in step 6) is reacted with an amine compound in the presence of a base to prepare a compound (1a) of Formula 1. In the case of N-hydroxyamidine (R₅═OH), hydroxylamine hydrochloride is reacted in the presence of a base, and the base can be selected from the group consisting of organic bases such as triethylamine, 1,8-diazabicyclo[5.4.0]undec-7-ene (DBU), diethylmethylamine (Et₂NMe), N-methylmorpholine, N-methylpiperidine, pyridine, and 2,6-dimethylpyridine, and inorganic bases such as potassium carbonate, potassium bicarbonate, sodium hydroxide, potassium hydroxide, sodium amide, sodium hydride, sodium methoxide, and sodium ethoxide. The reaction is preferably carried out at a temperature in the range of 60 to 90° C. for 1 to 15 hours. A single solvent such as methanol, ethanol and acetonitrile, or a mixed solvent thereof with water is preferably used as the reaction solvent.

In the case of amidine (R₅═H), methoxy imine is prepared from the reaction with a hydrochloride methanol solution at a temperature in the range of 10 to 30° C. for 24 to 48 hours, and then the solvent is removed under reduced pressure. The resultant is reacted with an ammonia ethanol solution at a temperature in the range of 45 to 60° C. for 24 to 50 hours in a high pressure reactor to prepare amidine. Ethanol is preferably used as the reaction solvent.

Reaction Scheme 2 will be described in detail as below.

In step 1), 4-(5-chloropentoxy)benzonitrile derivative (4) prepared in step 1) of Reaction Scheme 1 is reacted with 4-hydroxy propiophenone (13; R₃═H, X₁═O) in the presence of a base to prepare 4-[5-(4-propionyl-phenoxy)-pentyloxy]-benzonitrile (14). The base to be used for preparing the compound (14) may be an inorganic base, and preferably one selected from the group consisting of potassium carbonate, sodium hydroxide, and sodium hydride. The reaction is preferably carried out at a temperature in the range of 10 to 90° C. for 1 to 9 hours, and acetonitrile, dimethylformamide, or the like is preferably used as the reaction solvent.

In step 2), 4-[5-(4-propionyl-phenoxy)-pentyloxy]-benzonitrile (14) prepared in step 1) is reacted with a bromine compound to prepare an alpha-brominated compound, 4-{5-[4-(2-bromo-propionyl)-phenoxy]-pentyloxy}-benzonitrile (15). At this time, the reagent to be used for the reaction can be copper bromide (II) or bromine, and the reaction is preferably carried out at a temperature in the range of 20 to 80° C. for 8 to 24 hours, and ethyl acetate, dichloromethane, chloroform, or the like is used as the reaction solvent.

In step 3), the alpha-brominated compound (15) prepared in step 2) is reacted with the thioamide compound (8) to prepare a compound having a thiazole ring (16). The thioamide compound (8) to be used for the reaction is a substance to introduce the substituent R₁ into the compound of Formula 1, and the thioamide compound (8) with a proper substituent can be selected according to the type of the substituents. The reaction temperature and time may vary according to the type of the thioamide compound (8), and the reaction is preferably carried out at a temperature in the range of 60 to 90° C. for 5 to 24 hours. Examples of the thioamide compound (8) include thioacetamide, thiopropionamide, thioisobutyramide, trimethylthioacetamide, thiohexanoamide, cyclohexancarbothioicacidamide, N-(2-amino-2-thioxoethyl)-2-methylpropanamide, piperidine-4-carbothioicacidamide, thiourea, amidino thiourea, thiobenzamide, glycine thioamide, 2,2-dimethylthiopropionamide, N-methylthiourea, N-ethylthiourea, and N-propylthiourea, which are available commercially or simply synthesized by a method known in the art. Further, a single solvent such as ethanol, or a mixed solvent thereof with water is preferably used as the reaction solvent.

In step 4), the compound having a thiazole ring (16) prepared in step 3) is reacted with N-bromosuccinimide to prepare a compound (17). The reaction is preferably carried out at a temperature in the range of 0 to 80° C. for 1 to 4 hours, and carbon tetrachloride, chloroform, dichloromethane, or the like is preferably used as the reaction solvent.

In step 5), the compound (17) prepared in step 4) is reacted with a primary or secondary amine compound (11) to prepare a compound (18). The amine compound (11) to be used for preparing the compound (18) is a substance to introduce the substituent R₂ into the compound of Formula 1, and the amine compounds (11) can be suitably selected according to the type of the substituent. Examples of the amine compound (11) to be used include methylamine, dimethylamine, ethylamine, diethylamine, propylamine, isopropylamine, diisopropylamine, butylamine, dibutylamine, t-butylamine, isopropyloxypropylamine, piperidine, pyrrolidine, morpholine, pyrimidine, imidazole, N-methylpiperazine, N-methylethylamine, N,N-dimethylethylamine, dimethoxyethylamine, isobutyrylamine, dihydroxyethylamine, 2,6-dimethylmorpholine, thiomorpholine, aminoethylmorpholine, aminopropylimidazole, aminopropylmorpholine, aminoethylimidazole, cyclopentylamine, cyclopropylamine, and cyclohexylamine, which are commercially available, or can be simply synthesized for use by a method well known in the art. The reaction is preferably carried out at a temperature in the range of 20 to 180° C. for 1 to 24 hours. Further, acetonitrile, dimethylformamide, or the like is used as the reaction solvent, or the amine compound may be singly used without any solvent.

In step 6), the benzonitrile derivative (18) with a thiazole group substituted with a primary or secondary amine that is prepared in step 5) is reacted with an amine compound under the same condition and manner as in step 7) of Reaction Scheme 1 to prepare a compound of Formula 1b.

Reaction Scheme 3 will be described in detail as below.

In step 1), the 4-{5-[4-(2-bromo-acetyl)-phenoxy]-pentyloxy}-benzonitrile compound (7) prepared in step 3) of Reaction Scheme 1 is reacted with thiourea (19) to prepare a substituted compound (20) having an aminothiazole group.

In step 2), the substituted compound (20) having an aminothiazole group prepared in step 1) is reacted with bromine to prepare a compound (21). The reaction is preferably carried out at a temperature in the range of 0 to 80° C. for 1 to 4 hours. Further, chloroform, dichloromethane, ethyl acetate, or the like is preferably used as the reaction solvent.

In step 3), the compound (21) prepared in step 2) is reacted with the primary or secondary amine compound (11) to prepare a compound (22). The amine compound (11) to be used for preparing the compound (22) is a substance to introduce the substituent R₂ into the compound of Formula 1, and the amine compounds (11) can be suitably selected according to the type of the substituent. Examples of the amine compound (11) to be used include methylamine, dimethylamine, ethylamine, diethylamine, propylamine, isopropylamine, diisopropylamine, butylamine, dibutylamine, t-butylamine, isopropyloxypropylamine, piperidine, pyrrolidine, morpholine, pyrimidine, imidazole, N-methylpiperazine, N-methylethylamine, N,N-dimethylethylamine, dimethoxyethylamine, isobutyrylamine, dihydroxyethylamine, 2,6-dimethylmorpholine, thiomorpholine, aminoethylmorpholine, aminopropylimidazole, aminopropylmorpholine, aminoethylimidazole, cyclopentylamine, cyclopropylamine, and cyclohexylamine, which are commercially available, or can be simply synthesized for use by a method well known in the art. The reaction is preferably carried out at a temperature in the range of 20 to 180° C. for 1 to 24 hours. Further, acetonitrile, dimethylformamide, or the like is used as the reaction solvent, or the amine compound may be singly used without any solvent.

In step 4), the benzonitrile derivative (22) having a thiazole group prepared in step 3) is reacted with an amine compound under the same condition and manner as in step 7) of Reaction Scheme 1 to prepare a compound of Formula 1c.

Reaction Scheme 4 will be described in detail as below.

In step 1), the benzonitrile derivative having an aminothiazole group (20) prepared in step 1) of Reaction Scheme 3 is reacted with a halide compound (23) to prepare a compound (24). The halide compound (23) is a substance to introduce the substituent into the amino group of the compound (20), and the halide compound (23) having a proper substituent and halide can be suitably selected according to the type of the substituent. The reaction temperature and time may vary according to the type of the halide compound (23). The reaction is preferably carried out at a temperature in the range of 0 to 90° C. for 5 to 24 hours. Examples of the halide compound (23) include iodomethane, iodoethane, iodopropane, propyl bromide, 2-chloroethyl methyl ether, chloro ethyl morpholine, 3-bromomethylpyridine, bromoethanol, benzyl bromide, nicotinoyl chloride, ethanesulfonyl chloride, and isonicotinoyl chloride, which are commercially available, or can be simply synthesized by a method well known in the art. Dichloromethane, acetonitrile, dimethylformamide, or the like is preferably used as the reaction solvent.

In step 2), the benzonitrile derivative having a thiazole group (24) prepared in step 1) is reacted with formaldehyde and the amine compound (11) to prepare a compound (25). Examples of the amine compound (11) to be used for preparing the compound (25) include methylamine, dimethylamine, ethylamine, diethylamine, propylamine, isopropylamine, diisopropylamine butylamine, dibutylamine, t-butylamine, isopropyloxypropylamine, piperidine, pyrrolidine, morpholine, pyrimidine, imidazole, N-methylpiperazine, N-methylethylamine, N,N-dimethylethylamine, dimethoxyethylamine, isobutyrylamine, dihydroxyethylamine, 2,6-dimethylmorpholine, thiomorpholine, aminoethylmorpholine, aminopropylimidazole, aminopropylmorpholine, aminoethylimidazole, cyclopentylamine, cyclopropylamine, and cyclohexylamine, which are commercially available, or can be simply synthesized for use by a method well known in the art. The reaction is preferably carried out at a temperature in the range of 0 to 90° C. for 1 to 24 hours. Further, methanol, ethanol, acetonitrile, dimethylformamide, or the like is preferably used as the reaction solvent.

In step 3), the benzonitrile derivative having a thiazole group (25) prepared in step 2) is reacted with an amine compound under the same condition and manner as in step 7) of Reaction Scheme 1 to prepare a compound of Formula 1d.

Reaction Scheme 5 will be described in detail as below.

In step 1), the compound (9) prepared in step 4) of Reaction Scheme 1 is reacted with nitric acid to prepare a compound (26). The reaction is preferably carried out at a temperature in the range of 0 to 80° C. for 1 to 24 hours. Further, acetic acid, trifluoracetic acid, or the like is preferably used as the reaction solvent.

In step 2), the compound (26) prepared in step 1) is reacted with iron and ammonium chloride or tin chloride dihydrate to prepare a compound (27). The reaction is preferably carried out at a temperature in the range of 20 to 100° C. for 1 to 15 hours. A single solvent such as methanol, ethanol and acetonitrile, or a mixed solvent thereof with water is preferably used as the reaction solvent.

In step 3), the compound (27) prepared in step 2) is reacted with a halide compound (28) in the presence of a base to prepare a compound (29). The halide compound (28) is a substance to introduce the substituent into the amino group of the compound (27), and the halide compound (28) having a proper substituent and halide can be suitably selected according to the type of the substituent. The reaction temperature and time may vary according to the type of the halide compound (28). The reaction is preferably carried out at a temperature in the range of 0 to 90° C. for 1 to 24 hours. Examples of the halide compound (28) include iodomethane, iodoethane, iodopropane, propyl bromide, 2-chloroethyl methyl ether, chloro ethyl morpholine, 3-bromo methylpyridine, bromo ethanol, benzyl bromide, nicotinoyl chloride, ethanesulfonyl chloride, and isonicotinoyl chloride, which are commercially available, or can be simply synthesized for use by a method well known in the art. Further, dichloromethane, acetonitrile, dimethylformamide, or the like is preferably used as the reaction solvent.

In step 4), the compound (29) prepared in step 3) is reacted with an amine compound under the same condition and manner as in step 7) of Reaction Scheme 1 to prepare a compound of Formula 1e.

Reaction Scheme 6 will be described in detail as below.

In step 1), the compound (27) prepared in step 2) of Reaction Scheme 5 is reacted with a halide compound (28) in the presence of a base to prepare a compound (30). The halide compound (28) is a substance to introduce the substituent into the amino group of the compound (27), and the halide compound (28) having a proper substituent and halide can be suitably selected according to the type of the substituent. The reaction temperature and time may vary according to the type of the halide compound (28). The reaction is preferably carried out at a temperature in the range of 0 to 90° C. for 1 to 24 hours. Examples of the halide compound (28) include iodomethane, iodoethane, iodopropane, propyl bromide, 2-chloroethyl methyl ether, chloro ethyl morpholine, 3-bromo methylpyridine, bromo ethanol, benzyl bromide, nicotinoyl chloride, ethanesulfonyl chloride, and isonicotinoyl chloride, which are commercially available, or can be simply synthesized for use by a method well known in the art. Further, dichloromethane, acetonitrile, dimethylformamide, or the like is preferably used as the reaction solvent.

In step 2), the compound (30) prepared in step 1) is reacted with an amine compound under the same condition and manner as in step 7) of Reaction Scheme 1 to prepare a compound of Formula 1f.

Reaction Scheme 7 will be described in detail as below.

In step 1), the compound (7) prepared in step 3) of Reaction Scheme 1 is reacted with a primary or secondary amine compound (11) to prepare a compound (31). The amine compound (11) is a substance to introduce the substituent R₂ into the compound of Formula 7, and the amine compounds (11) can be suitably selected according to the type of the substituent. Examples of the amine compound (11) to be used include methylamine, dimethylamine, ethylamine, diethylamine, propylamine, isopropylamine, diisopropylamine, butylamine, dibutylamine, t-butylamine, isopropyloxypropylamine, piperidine, pyrrolidine, morpholine, pyrimidine, imidazole, N-methylpiperazine, N-methylethylamine, N,N-dimethylethylamine, dimethoxyethylamine, isobutyrylamine, dihydroxyethylamine, 2,6-dimethylmorpholine, thiomorpholine, aminoethylmorpholine, aminopropylimidazole, aminopropylmorpholine, aminoethylimidazole, cyclopentylamine, cyclopropylamine, and cyclohexylamine, which are commercially available, or can be simply synthesized for use by a method well known in the art. The reaction is preferably carried out at a temperature in the range of 0 to 100° C. for 1 to 24 hours. Further, dichloromethane, chloroform, acetonitrile, tetrahydrofuran, dimethylformamide, dimethylsulfoxide is preferably used as the reaction solvent.

In step 2), the compound (31) prepared in step 1) is reacted with a bromine compound to prepare an alpha-brominated compound (32). At this time, the reagent to be used for the reaction can be copper bromide (II) or bromine, and the reaction is preferably carried out at a temperature in the range of 0 to 80° C. for 1 to 15 hours, and dichloromethane, chloroform, ethyl acetate, or the like is used as the reaction solvent.

In step 3), the compound (32) prepared in step 2) is reacted with a thioamide compound (8) to prepare a compound having a thiazole ring (12). The thioamide compound (8) to be used for the reaction is a substance to introduce the substituent R₁ into the compound of Formula 1, and the thioamide compound (8) can be suitably selected according to the type of the substituent. The reaction temperature and time may vary according to the type of the thioamide compound (8), and the reaction is preferably carried out at a temperature in the range of 60 to 90° C. for 5 to 24 hours. Examples of the thioamide compound (8) include thioacetamide, thiopropionamide, thioisobutyramide, trimethylthioacetamide, thiohexanoamide, cyclohexancarbothioicacidamide, N-(2-amino-2-thioxoethyl)-2-methylpropanamide, piperidine-4-carbothioicacidamide morpholin-4-carbothioicacidamide, thiourea, amidino thiourea, thiobenzamide, glycine thioamide, 2,2-dimethylthiopropionamide, N-methylthiourea, N-ethylthiourea, and N-propyl thiourea, which are commercially available, or can be simply synthesized for use by a method well known in the art. Further, a single solvent such as ethanol, or a mixed solvent thereof with water is used as the reaction solvent.

In step 4), the compound (12) prepared in step 3) is reacted with an amine compound under the same condition and manner as in step 7) of Reaction Scheme 1 to prepare a compound of Formula 1a.

Reaction Scheme 8 will be described in detail as below.

In step 1), the compound (27) prepared in step 2) of Reaction Scheme 5 is reacted with an amine compound under the same condition and manner as in step 7) of Reaction Scheme 1 to prepare a compound of Formula 1g.

Reaction Scheme 9 will be described in detail as below.

In step 1), the benzonitrile derivative having a thiazole group (9) prepared in step 4) of Reaction Scheme 1 is reacted with formaldehyde and the amine compound (11) to prepare a compound (18). Examples of the amine compound (11) to be used for preparing the compound (18) include methylamine, dimethylamine, ethylamine, diethylamine, propylamine, isopropylamine, diisopropylamine, butylamine, dibutylamine, t-butylamine, isopropyloxypropylamine, piperidine, pyrrolidine, morpholine, pyrimidine, imidazole, N-methylpiperazine, N-methylethylamine, N,N-dimethylethylamine, dimethoxyethylamine, isobutyrylamine, dihydroxyethylamine, 2,6-dimethylmorpholine, thiomorpholine, aminoethylmorpholine, aminopropylimidazole, aminopropylmorpholine, aminoethylimidazole, cyclopentylamine, cyclopropylamine, and cyclohexylamine, which are commercially available, or can be simply synthesized for use by a method well known in the art. The reaction is preferably carried out at a temperature in the range of 0 to 90° C. for 1 to 24 hours. Further, methanol, ethanol, acetonitrile, dimethylformamide, or the like is preferably used as the reaction solvent.

In step 2), the benzonitrile derivative having a thiazole group (18) prepared in step 1) is reacted with an amine compound under the same condition and manner as in step 7) of Reaction Scheme 1 to prepare a compound of Formula 1b.

Reaction Scheme 10 will be described in detail as below.

In step 1), the 4-{5-[4-(2-bromo-acetyl)-phenoxy]-pentyloxy}-benzonitrile compound (7) prepared in step 3) of Reaction Scheme 1 is reacted with thiourea to prepare a compound having an aminothiazole ring (33).

In step 2), the benzonitrile derivative having an aminothiazole ring (33) prepared in step 1) is reacted with a compound (34), of which both terminals are substituted with halogen, in the presence of a base to prepare a benzonitrile derivative (35) having a thiazole group, in which R₁ substituted with a heteroring. The compound (34), of which both terminals are substituted with halogen, is a substance to introduce

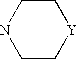

into the substituent R₁ in the compound of Formula 1, and the compound (34) can be suitably selected according to the type of the substituent. The reaction is preferably carried out at a temperature in the range of 0 to 90° C. for 4 to 24 hours. Examples of the compound (34) include mechlorethylamine, bis-dibromide ethylester, and 1,5-dibromopentane, which are commercially available, or can be simply synthesized for use by a method well known in the art. Further, acetonitrile, dimethylformamide, or the like is preferably used as the reaction solvent.

In step 3), the compound having a thiazole ring (35) prepared in step 2) is reacted with bromine to prepare a compound (36). The reaction is preferably carried out at a temperature in the range of 0 to 80° C. for 1 to 4 hours. Chloroform, dichloromethane, ethyl acetate, or the like is preferably used as the reaction solvent.

In step 4), the compound (36) prepared in step 3) is reacted with the primary or secondary amine compound (11) to prepare a compound (37). The amine compound (11) to be used for preparing the compound (37) is a substance to introduce the substituent R₂ into the compound of Formula 1, and the amine compounds (11) can be suitably selected according to the type of the substituent. Examples of the amine compound (11) include methylamine, dimethylamine, ethylamine, diethylamine, propylamine, isopropylamine, diisopropylamine, butylamine, dibutylamine, t-butylamine, isopropyloxypropylamine, piperidine, pyrrolidine, morpholine, pyrimidine, imidazole, N-methylpiperazine, N-methylethylamine, N,N-dimethylethylamine, dimethoxyethylamine, isobutyrylamine, dihydroxyethylamine, 2,6-dimethylmorpholine, thiomorpholine, aminoethylmorpholine, aminopropylimidazole, aminopropylmorpholine, aminoethylimidazole, cyclopentylamine, cyclopropylamine, and cyclohexylamine, which are commercially available, or can be simply synthesized for use by a method well known in the art. The reaction is preferably carried out at a temperature in the range of 20 to 180° C. for 1 to 24 hours. Further, acetonitrile, dimethylformamide, or the like is preferably used as the reaction solvent, or the amine compound may be singly used without any solvent.

In step 5), the benzonitrile derivative having a thiazole group (37) prepared in step 4) is reacted with an amine compound under the same condition and manner as in step 7) of Reaction Scheme 1 to prepare a compound of Formula 1h.

In accordance with still another aspect, the present invention relates to a pharmaceutical composition for preventing or treating osteoporosis, comprising the compound of Formula 1 or a pharmaceutically acceptable salt thereof.

The term “osteoporosis” as used herein means the state that minerals and substrates forming the bone are reduced in abnormally large amounts, even without any defect in the structure of the remaining bone, so that many pores are generated in the bone, making it spongelike and more likely to fracture. This may be referred to as “osteopenia”. In specific embodiments, the benzamidine derivative of Formula 1 of the present invention suppresses the differentiation of osteoclast at a low concentration.

The composition of the present invention may comprise one or more effective ingredients which are equivalent or similar in function to the benzamidine derivative, in addition to the benzamidine derivative or a pharmaceutically acceptable salt thereof.

The composition of the present invention may be prepared by adding one or more pharmaceutically acceptable carriers in addition to the above-described ingredients. The pharmaceutically acceptable carrier may be saline, sterilized water, a Ringer's solution, buffered saline, a dextrose solution, a maltodextrin solution, glycerol, ethanol, and a combination thereof, and may be, if necessary, further supplemented with other typical additives such as an antioxidant, a buffer and a static agent. In combination with a diluent, a dispersant, a surfactant, a binder, and a lubricant, the composition of the present invention may be also formulated into injectable dosage forms such as an aqueous solution, a suspension, and an emulsion, pills, capsules, granules, or tablets. Moreover, depending on the kind of the ingredient or the disease, the formulation may be preferably prepared using a method known in the art or disclosed in Remington's Pharmaceutical Science (latest version), Mack Publishing Company, Easton Pa.

The composition of the present invention may be administered orally or parenterally (e.g., intravenously, subcutaneously, intraperitoneally, or topically). The dosage varies depending on the body weight, age, gender, health state, diet, administration time, administration route, excretion rate, and disease severity of a patient. The benzamidine derivative is administered once or several times at a daily dose of approximately 5 to 1,000 mg/kg, and preferably at a daily dose of approximately 10 to 500 mg/kg.

For the prevention and treatment of osteoporosis, the composition of the present invention may be used alone or in combination with surgical operations, hormone therapies, chemical therapies, and other methods using biological reaction regulators.

### ADVANTAGEOUS EFFECTS

The benzamidine derivatives of the present invention effectively inhibit osteoclast differentiation at an extremely low concentration, and thus it can be advantageously used for the prevention and treatment of osteoporosis.

### MODE FOR INVENTION

A better understanding of the present invention may be obtained through the following preferable Examples and Experimental Examples, which are set forth to illustrate, but are not to be construed as the limit of the present invention.

### Preparative Example 1

### Preparation of Compound (12) in Reaction Scheme 1

### 1-1: 4-(5-chloropentoxy)-benzonitrile (4)

3.0 g (25.2 mmol) of 4-cyanophenol and 3.67 g (27 mmol) of potassium carbonate were sequentially added to 80 ml of acetonitrile, and then 4.67 g (25.2 mmol) of 1-bromo-5-chloropentane was added thereto. Subsequently, the mixture was refluxed for 7 hrs while maintaining the temperature at 80 to 82° C., and then cooled to room temperature after stopping heating. The reaction solution was diluted with ethyl acetate, and washed with purified water, and then the organic layer was dried over anhydrous magnesium sulfate. The solvent was distilled off under reduced pressure, and the residue was recrystallized from methanol, and then washed with methanol at −10° C. The resultant was dried under reduced pressure to obtain 5.09 g (yield: 90.3%) of a title compound (4).

¹H-NMR (CDCl₃) (ppm) 1.64 (m, 2H), 1.82 (m, 4H), 3.57 (t, 2H), 4.01 (t, 2H), 6.93 (d, 2H), 7.57 (d, 2H).

### 1-2: 4-[5-(4-acetyl-phenoxy)-pentyloxy]-benzonitrile (6)

30.0 g (220 mmol) of 4-hydroxyacetophenone was added to and dissolved in 0.1 L of N,N-dimethylformamide, and 36.5 g (264 mmol) of potassium carbonate was slowly added to the solution. The mixture was warmed to 50° C., and then stirred for 1 hr. 53.3 g (225 mmol) of 4-(5-chloropentoxy)-benzonitrile obtained in the above 1-1 was added thereto at the same temperature, and the mixture was warmed to 95° C., and then stirred for 5 hrs. The reaction solution was cooled to room temperature, and diluted with ethyl acetate, and the organic layer was washed with water and a sodium chloride solution. The organic layer was dried over anhydrous magnesium sulfate, recrystallized from methanol, and dried under reduced pressure to obtain 63.0 g (yield: 88%) of a title compound (6).

¹H-NMR (DMSO-d₆) (ppm) 1.56 (m, 2H), 1.80 (m, 4H), 2.51 (s, 3H), 4.08 (m, 4H), 7.02 (d, 2H), 7.09 (d, 2H), 7.75 (d, 2H), 7.92 (d, 2H).

### 1-3: 4-{5-[4-(2-bromo-acetyl)-phenoxy]-pentyloxy}-benzonitrile (7)

63.0 g (195 mmol) of the 4-[5-(4-acetyl-phenoxy)-pentyloxy]-benzonitrile compound (6) obtained in the above 1-2 was dissolved in 200 ml of ethyl acetate, and 87.0 g (390 mmol) of copper (II) bromide was added thereto. The mixture was refluxed at a temperature of 70° C. for 8 hrs. The reaction solution was cooled to room temperature, and then the salts generated during the reaction were filtered off, and the ethyl acetate layer was washed with a sodium bicarbonate solution and a sodium chloride solution. The organic layer was dried over anhydrous magnesium sulfate, recrystallized from methanol, and then dried under reduced pressure to obtain 62.6 g (yield: 80%) of a title compound (7).

¹H-NMR (DMSO-d₆) (ppm) 1.57 (m, 2H), 1.79 (m, 4H), 4.08 (m, 4H), 4.83 (s, 2H), 7.07 (m, 4H), 7.75 (d, 2H), 7.97 (d, 2H).

### 1-4: 4-{5-[4-(2-methyl-thiazol-4-yl)-phenoxy]-pentyloxy}-benzonitrile (9)

40.0 g (99.4 mmol) of the 4-{5-[4-(2-bromo-acetyl)-phenoxy]-pentyloxy}-benzonitrile compound (7) obtained in the above 1-3 was added to 150 ml of ethanol, and then 14.9 g (199 mmol) of thioacetamide was added thereto. The mixture was refluxed at a temperature of 80° C. for 12 hrs. The reaction solution was cooled to room temperature, diluted with ethyl acetate, and then washed with a sodium bicarbonate solution and a sodium chloride solution. The organic layer was dried over anhydrous magnesium sulfate, recrystallized from methanol, and then dried under reduced pressure to obtain 25.5 g (yield: 68%) of a title compound (9).

¹H-NMR (CDCl₃) (ppm) 1.58 (m, 2H), 1.80 (m, 4H), 2.69 (s, 3H), 4.02 (m, 2H), 4.08 (m, 2H), 6.97 (d, 2H), 7.10 (d, 2H), 7.73 (s, 1H), 7.75 (d, 2H), 7.83 (d, 2H).

### 1-5: 4-{5-[4-(5-bromo-2-methyl-thiazol-4-yl)-phenoxy]-pentyloxy}-benzonitrile (10)

13 g (34 mmol) of 4-{5-[4-(2-methyl-thiazol-4-yl)-phenoxy]-pentyloxy}-benzonitrile (9) obtained in the above 1-4 was added to 120 ml of chloroform, and then 1.8 mL (34 mmol) of bromine diluted in 12 mL of chloroform was slowly added thereto. The mixture was stirred at room temperature for 3 hours. The reaction solution was diluted with dichloromethane, and then washed with a sodium bisulfite solution and a sodium chloride solution. The organic layer was dried over anhydrous magnesium sulfate, and then dried under reduced pressure to obtain 15 g (yield: 92%) of a title compound (10).

¹H-NMR (DMSO-d₆) (ppm) 1.58 (m, 2H), 1.79 (m, 4H), 2.65 (s, 3H), 4.06 (m, 4H), 7.01 (d, 2H), 7.09 (d, 2H), 7.74 (d, 2H), 7.81 (d, 2H).

### 1-6: 4-{5-[4-(2-methyl-5-morpholin-4-yl-thiazol-4-yl)-phenoxy]-pentyloxy}-benzonitrile (12)

10 ml of morpholine was added to 1.1 g (24 mmol) of 4-{5-[4-(5-bromo-2-methyl-thiazol-4-yl)-phenoxy]-pentyloxy}-benzonitrile (10) obtained in the above 1-5, and then stirred at 120° C. for 22 hours. The reaction solution was cooled to room temperature, diluted with ethyl acetate, and then washed with purified water and a sodium chloride solution. The organic layer was dried over anhydrous magnesium sulfate, the solvent was removed therefrom, and the residue was purified by column chromatography to obtain 170 mg (yield: 15%) of a title compound (12).

¹H-NMR (DMSO-d₆) (ppm) 1.58 (m, 2H), 1.79 (m, 4H), 2.59 (s, 3H), 2.78 (m, 4H), 3.73 (m, 4H), 4.01 (m, 4H), 6.95 (m, 4H), 7.58 (d, 2H), 8.05 (d, 2H).

### Preparative Example 2

### Preparation of Compound (18) in Reaction Scheme 2

### 2-1: 4-[5-(4-propionyl-phenoxy)-pentyloxy]-benzonitrile (14)

30.0 g (200 mmol) of 4-hydroxypropiophenone was added to and dissolved in 0.1 L of N,N-dimethylformamide, and 9.59 g (240 mmol) of sodium hydroxide was slowly added thereto. The temperature was increased to 70° C., and then the mixture was stirred for 1 hour. 45.6 g (204 mmol) of 4-(5-chloropentoxy)-benzonitrile (4) obtained in Preparative Example 1-1 was added thereto at the same temperature, and the temperature was increased to 95° C., followed by stirring for 5 hours. The reaction solution was cooled to room temperature, diluted with ethyl acetate, and then the organic layer was washed with water and a sodium chloride solution. The organic layer was dried over anhydrous magnesium sulfate, recrystallized from methanol, and then dried under reduced pressure to obtain 56.9 g (yield: 84%) of a title compound (14).

¹H-NMR (DMSO-d₆) (ppm) 1.06 (t, 3H), 1.57 (m, 2H), 1.79 (m, 4H), 2.95 (m, 2H), 4.08 (m, 4H), 7.02 (d, 2H), 7.09 (d, 2H), 7.74 (d, 2H), 7.91 (d, 2H).

### 2-2: 4-{5-[4-(2-bromo-propionyl)-phenoxy]-pentyloxy}-benzonitrile (15)

20.0 g (59.3 mmol) of 4-[5-(4-propionyl-phenoxy)-pentyloxy]-benzonitrile (14) obtained in 2-1 was dissolved in 100 ml of ethyl acetate, and 26.5 g (119 mmol) of copper bromide (II) was added thereto. The mixture was refluxed at a temperature of 70° C. for 8 hrs. The reaction solution was cooled to room temperature, and then the salts generated during the reaction were filtered off, and the ethyl acetate layer was washed with a sodium bicarbonate solution and a sodium chloride solution. The organic layer was dried over anhydrous magnesium sulfate, recrystallized using ethyl acetate and n-hexane, and then dried under reduced pressure to obtain 19.7 g (yield: 80%) of a title compound (15).

¹H-NMR (DMSO-d₆) (ppm) 1.57 (m, 2H), 1.73 (d, 3H), 1.78 (m, 4H), 4.09 (m, 4H), 5.76 (q, 1H), 7.07 (m, 4H), 7.74 (d, 2H), 7.98 (d, 2H).

### 2-3: 4-{5-[4-(2,5-dimethyl-thiazol-4-yl)-phenoxy]-pentyloxy}-benzonitrile (16)

5.07 g (12.2 mmol) of 4-{5-[4-(2-bromo-propionyl)-phenoxy]-pentyloxy}-benzonitrile (15) obtained in the above 2-2 was added to 50 ml of ethanol, and then 1.83 g (24.4 mmol) of thioacetamide was added thereto. The mixture was refluxed at a temperature of 80° C. for 12 hrs. The reaction solution was cooled to room temperature, diluted with ethyl acetate, and then washed with a sodium bicarbonate solution and a sodium chloride solution. The organic layer was dried over anhydrous magnesium sulfate, recrystallized from methanol, and then dried under reduced pressure to obtain 3.59 g (yield: 75%) of a title compound (16).

¹H-NMR (CDCl₃) (ppm) 1.57 (m, 2H), 1.78 (m, 4H), 2.44 (s, 3H), 2.58 (s, 3H), 4.01 (m, 4H), 6.97 (m, 4H), 7.54 (d, 2H), 7.57 (d, 2H).

### 2-4: 4-{5-[4-(5-bromomethyl-2-methyl-thiazol-4-yl)-phenoxy]-pentyloxy}-benzonitrile (17)

40 ml of carbon tetrachloride was added to 3.59 g (9.15 mmol) of 4-{5-[4-(2,5-dimethyl-thiazol-4-yl)-phenoxy]-pentyloxy}-benzonitrile (16) obtained in the above 2-3, and then 1.79 g (10.1 mmol) of N-bromosuccinimide and 150 mg (0.915 mmol) of 2,2′-azo bisisobutyronitrile (AIBN) were added thereto. The mixture was refluxed for 4 hrs. The reaction solution was cooled to room temperature, diluted with ethyl acetate, and then washed with a sodium bicarbonate solution and a sodium chloride solution. The organic layer was dried over anhydrous magnesium sulfate, the solvent was removed therefrom, and then dried under reduced pressure to obtain 3.87 g (yield: 90%) of a title compound (17).

¹H-NMR (DMSO-d₆) (ppm) 1.57 (m, 2H), 1.80 (m, 4H), 2.46 (s, 2H), 2.68 (s, 3H), 4.08 (m, 4H), 7.02 (d, 2H), 7.09 (d, 2H), 7.54 (d, 2H), 7.74 (d, 2H).

### 2-5: 4-{5-[4-(2-methyl-5-morpholin-4-ylmethyl-thiazol-4-yl)-phenoxy]-pentyloxy}-benzonitrile (18)

10 ml of acetonitrile and 0.18 ml of morpholine were added to 500 mg (1.1 mmol) of 4-{5-[4-(5-bromomethyl-2-methyl-thiazol-4-yl)-phenoxy]-pentyloxy}-benzonitrile (17) obtained in the above 2-4, and then refluxed for 1 hr. The reaction solution was cooled to room temperature, diluted with ethyl acetate, and then washed with purified water and a sodium chloride solution. The organic layer was dried over anhydrous magnesium sulfate, the solvent was removed therefrom, and then purified by column chromatography to obtain 180 mg (yield: 35%) of a title compound (18).

¹H-NMR (DMSO-d₆) (ppm) 1.59 (m, 2H), 1.80 (m, 4H), 2.51 (m, 4H), 3.34 (s, 3H), 3.61 (m, 4H), 3.77 (s, 2H), 4.03 (m, 4H), 6.92 (d, 2H), 7.01 (d, 2H), 7.58 (m, 4H).

### Preparative Example 3

### Preparation of Compound (22) in Reaction Scheme 3

### 3-1: 4-{5-[4-(2-methylamino-thiazol-4-yl)-phenoxy]-pentyloxy}-benzonitrile (20)

1.98 g (4.92 mmol) of 4-{5-[4-(2-bromo-acetyl)-phenoxy]-pentyloxy}-benzonitrile (7) obtained in Preparative Example 1-3 was added to 20 ml of ethanol, and then 488 mg (5.41 mmol) of N-methylthiourea was added thereto. The mixture was refluxed at a temperature of 80° C. for 2 hrs. The reaction solution was cooled to room temperature, recrystallized from water, washed with ethyl acetate, and then dried under reduced pressure to obtain 1.74 g (yield: 90%) of a title compound (20).

¹H-NMR (DMSO-d₆) (ppm) 1.58 (m, 2H), 1.79 (m, 4H), 2.87 (s, 3H), 4.00-4.09 (m, 4H), 6.89 (s, 1H), 6.93 (d, 2H), 7.10 (d, 2H), 7.76 (m, 4H).

### 3-2: 4-{5-[4-(5-bromo-2-methylamino-thiazol-4-yl)-phenoxy]-pentyloxy}-benzonitrile (21)

30 ml of chloroform was added to 3.0 g (7.6 mmol) of 4-{5-[4-(2-methylamino-thiazol-4-yl)-phenoxy]-pentyloxy}-benzonitrile (20) obtained in the above 3-1, and then 0.40 ml (7.6 mmol) was added thereto. The mixture was stirred at room temperature for 1 hr. The solvent was removed from the reaction solution, and then the resultant was used as a starting material.

### 3-3: 4-{5-[4-(2-methylamino-5-morpholin-4-yl-thiazol-4-yl)-phenoxy]-pentyloxy}-benzonitrile (22)

13 ml of morpholine was added to 4-{5-[4-(5-bromo-2-methylamino-thiazol-4-yl)-phenoxy]-pentyloxy}-benzonitrile (21) obtained in the above 3-2, and stirred at 120° C. for 1 hr. The reaction solution was cooled to room temperature, diluted with ethyl acetate, and then washed with a sodium bicarbonate solution and a sodium chloride solution. The organic layer was dried over anhydrous magnesium sulfate, the solvent was removed therefrom, and then purified by column chromatography to obtain 970 mg (yield: 27%) of a title compound (22).

¹H-NMR (DMSO-d₆) (ppm) 1.58 (m, 2H), 1.80 (m, 4H), 2.72 (m, 4H), 3.34 (s, 3H), 3.71 (m, 4H), 4.01 (m, 4H), 6.92 (m, 4H), 7.60 (d, 2H), 7.92 (d, 2H).

### Preparative Example 4

### Preparation of Compound (25) in Reaction Scheme 4

### 4-1: 4-[5-(4-{2-[methyl-(2-morpholin-4-yl-ethyl)-amino]-thiazol-4-yl}-phenoxy)-pentyloxy]-benzonitrile (24)

100 ml of dimethylsulfoxide was added to, and dissolved in 10.0 g (25.4 mmol) of 4-{5-[4-(2-methylamino-thiazol-4-yl)-phenoxy]-pentyloxy}-benzonitrile (20) obtained in Preparative Example 3-1. 3.05 g (76.24 mmol) of sodium hydride and 5.67 g (30.5 mmol) of N-(2-chloroethyl)morpholine hydrochloride were added thereto. The mixture was stirred at 50° C. for 4 hrs. The reaction solution was cooled to room temperature, diluted with ethyl acetate, and then washed with purified water. The organic layer was dried over anhydrous magnesium sulfate, the solvent was removed therefrom, and purified by column chromatography to obtain 7.16 g (yield: 56%) of a title compound (24)

¹H-NMR (DMSO-d₆) (ppm) 1.57 (m, 2H), 1.79 (m, 4H), 2.45 (m, 2H), 2.51 (m, 4H), 3.07 (s, 3H), 3.55 (m, 4H), 3.62 (m, 2H), 4.00-4.09 (m, 4H), 6.94 (s, 1H), 6.96 (d, 2H), 7.11 (d, 2H), 7.76 (m, 4H).

### 4-2: 4-[5-(4-{2-[methyl-(2-morpholin-4-yl-ethyl)-amino]-5-morpholine-4-ylmethyl-thiazol-4-yl}-phenoxy)-pentyloxy]-benzonitrile (25)

30 ml of ethanol was added to 5.00 g (9.87 mmol) of 4-[5-(4-{2-[methyl-(2-morpholin-4-yl-ethyl)-amino]-thiazol-4-yl}-phenoxy)-pentyloxy]-benzonitrile (24) obtained in the above 4-1, and then 7.6 ml (98.7 mmol) of formaldehyde (35%) and 7.7 ml (88.8 mmol) of morpholine were added thereto. The mixture was refluxed at 80° C. for 2 hrs. The reaction solution was cooled to room temperature, diluted with ethyl acetate, and then washed with brine. The organic layer was dried over anhydrous magnesium sulfate, the solvent was removed therefrom, and purified by column chromatography to obtain 3.81 g (yield: 64%) of a title compound (25).

¹H-NMR (DMSO-d₆) (ppm) 1.57 (m, 2H), 1.79 (m, 4H), 2.43-2.51 (m, 8H), 2.55 (m, 1H), 3.02 (s, 3H), 3.16 (m, 1H), 3.53-3.56 (m, 12H), 4.01-4.11 (m, 4H), 6.95 (d, 2H), 7.10 (d, 2H), 7.49 (d, 2H), 7.76 (d, 2H).

### Preparative Example 5

### Preparation of Compound (26) in Reaction Scheme 5

### 5-1: 4-{5-[4-(2-methyl-5-nitro-thiazol-4-yl)-phenoxy-pentyloxy}-benzonitrile (26)

12.9 g (34.0 mmol) of 4-{5-[4-(2-methyl-thiazol-4-yl)-phenoxy]-pentyloxy}-benzonitrile (9) obtained in Preparative Example 1-4 was dissolved in 130 ml of acetic acid, and 2.30 ml of 65% nitric acid was added thereto. The temperature was increased to 80° C., and the mixture was stirred for 3 hrs. The reaction solution was diluted with ethyl acetate, and then washed with purified water and a sodium chloride solution. The organic layer was dried over anhydrous magnesium sulfate, the solvent was removed therefrom, recrystallized from methanol at 0° C., and then dried under reduced pressure to obtain 13 g (yield: 90%) of a title compound (26).

¹H-NMR (DMSO-d₆) (ppm) 1.59 (m, 2H), 1.81 (m, 4H), 2.71 (s, 3H), 4.09 (m, 4H), 7.03 (d, 2H), 7.10 (d, 2H), 7.73 (d, 2H), 7.75 (d, 2H).

### 5-2: 4-{5-[4-(5-amino-2-methyl-thiazol-4-yl)-phenoxy-pentyloxy}-benzonitrile (27)

A mixed solvent of water and ethanol (1:1) was added to 1.20 g (2.83 mmol) of 4-{5-[4-(2-methyl-5-nitro-thiazol-4-yl)-phenoxy-pentyloxy}-benzonitrile (26) obtained in Preparative Example 5-1, and 790 mg (14.1 mmol) of iron and 30 mg (0.57 mmol) of ammonium chloride were added thereto. The mixture was refluxed for 8 hrs. The reaction solution was diluted with dichloromethane, and then washed with a sodium bicarbonate solution and a sodium chloride solution. The organic layer was dried over anhydrous magnesium sulfate, the solvent was removed therefrom, and then purified by column chromatography to obtain 500 mg (yield: 45%) of a title compound (27).

¹H-NMR (DMSO-d₆) (ppm) 1.57 (m, 2H), 1.79 (m, 4H), 2.44 (s, 3H), 3.99 (t, 2H), 4.08 (t, 2H), 5.35 (s, 2H), 6.91 (d, 2H), 7.10 (d, 2H), 7.66 (d, 2H), 7.75 (d, 2H).

### 5-3: 4-{5-[4-(2-methyl-5-methylamino-thiazol-4-yl)-phenoxy]-pentyloxy}-benzonitrile (29)

3.0 (7.6 mmol) 4-{5-[4-(5-amino-2-methyl-thiazol-4-yl)-phenoxy-pentyloxy}-benzonitrile compound (27) obtained in Preparative Example 5-2 and 590 mg (15 mmol) of sodium hydride were added to 60 ml of N,N-dimethylformamide, and then the mixture was stirred at room temperature for 30 min. 0.91 ml (15 mmol) of methyl iodide was added to the reaction solution at the same temperature, and stirred for 30 min. The reaction solution was diluted with ethylacetate, and then washed with purified water. The organic layer was dried over anhydrous magnesium sulfate, the solvent was removed therefrom, purified by column chromatography and dried under reduced pressure to obtain 1.6 g (yield: 51%) of a title compound (29).

¹H-NMR (DMSO-d₆) (ppm) 1.58 (m, 2H), 1.79 (m, 4H), 2.50 (s, 3H), 2.61 (s, 3H), 4.04 (m, 4H), 6.92 (m, 4H), 7.58 (d, 2H), 7.89 (d, 1H), 7.97 (d, 1H).

### Preparative Example 6

### Preparation of Compound (30) in Reaction Scheme 6

### 6-1: 4-{5-[4-(5-dimethylamino-2-methyl-thiazol-4-yl)-phenoxy]-pentyloxy}-benzonitrile (30)

1.00 g (2.54 mmol) of 4-{5-[4-(5-amino-2-methyl-thiazol-4-yl)-phenoxy-pentyloxy}-benzonitrile compound (27) obtained in Preparative Example 5-2 was dissolved in 15 ml of N,N-dimethylformamide, and then 128 mg (5.33 mmol) of sodium hydride and 0.32 ml (5.08 mmol) of methyliodide were added thereto. The mixture was stirred at 70° C. for 2 hrs. The reaction solution was diluted with ethyl acetate, and then washed with purified water. The organic layer was dried over anhydrous magnesium sulfate, the solvent was removed therefrom, purified by column chromatography and dried under reduced pressure to obtain 400 mg (yield: 37%) of a title compound (30).

¹H-NMR (DMSO-d₆) (ppm) 1.57 (m, 2H), 1.79 (m, 4H), 2.50 (s, 3H), 2.57 (s, 6H), 4.00 (t, 2H), 4.08 (t, 2H), 6.94 (d, 2H), 7.00 (d, 2H), 7.74 (d, 2H), 7.97 (d, 2H).

### Preparative Example 7

### Preparation of Compound (12) in Reaction Scheme 7

### 7-1: 4-{5-[4-(2-[1,2,4]triazol-1-yl-acetyl)-phenoxy-pentyloxy}-benzonitrile (31)

3.0 g (7.5 mmol) of 4-{5-[4-(2-bromo-acetyl)-phenoxy]-pentyloxy}-benzonitrile compound (7) obtained in Preparative Example 1-3 was dissolved in 70 ml of acetonitrile, and then 680 mg (7.5 mmol) of 1,2,4-triazole sodium was added thereto. The mixture was stirred at room temperature for 18 hrs. The reaction solution was diluted with ethyl acetate, and then washed with purified water and a sodium chloride solution. The organic layer was dried over anhydrous magnesium sulfate, and separated by column chromatography to obtain 1.7 g (yield: 59%) of a title compound (31).

¹H-NMR (DMSO-d₆) (ppm) 1.58 (m, 2H), 1.82 (m, 4H), 4.11 (m, 4H), 5.92 (s, 2H), 7.10 (m, 4H), 7.76 (d, 2H), 8.00 (s, 1H), 8.02 (d, 2H), 8.50 (s, 1H).

### 7-2: 4-{5-[4-(2-bromo-2-[1,2,4]triazol-1-yl-acetyl)-phenoxy-pentyloxy}-benzonitrile (32)

850 mg (2.2 mmol) of 4-{5-[4-(2-[1,2,4]triazol-1-yl-acetyl)-phenoxy-pentyloxy}-benzonitrile (31) obtained in the above 7-1 was dissolved in 5 ml of acetic acid, and 180 mg (2.2 mmol) of sodium acetic acid was added thereto. The temperature was increased to 40° C., and 0.11 ml of (2.2 mmol) of bromine was added thereto, followed by stirring at the same temperature for 30 min. The reaction solution was cooled to room temperature, diluted with dichloromethane, and washed with a sodium bicarbonate solution and a sodium chloride solution. The organic layer was dried over anhydrous magnesium sulfate, and dried under reduced pressure to obtain 880 mg (yield: 88%) of a title compound (32).

¹H-NMR (DMSO-d₆) (ppm) 1.59 (m, 2H), 1.80 (m, 4H), 4.09 (m, 4H), 7.10 (m, 4H), 7.34 (s, 1H), 7.75 (d, 2H), 8.01 (s, 1H), 8.03 (d, 2H), 8.49 (s, 1H).

### 7-3: 4-{5-[4-(2-methyl-5-[1,2,4]triazol-1-yl-thiazol-4-yl)-phenoxy-pentyloxy}-benzonitrile (12)

880 mg (1.9 mmol) of 4-{5-[4-(2-bromo-2-[1,2,4]triazol-1-yl-acetyl)-phenoxy-pentyloxy}-benzonitrile (32) obtained in the above 7-2 was added to 10 ml of ethanol, and 280 mg (3.8 mmol) of thioacetamide was added thereto. The mixture was refluxed at 80° C. for 6 hrs. The reaction solution was cooled to room temperature, diluted with ethyl acetate, and washed with a potassium carbonate solution and a sodium chloride solution. The organic layer was dried over magnesium sulfate, and purified by column chromatography to obtain 60 mg (yield: 7%) of a title compound (12).

¹H-NMR (DMSO-d₆) (ppm) 1.56 (m, 2H), 1.77 (m, 4H), 2.76 (s, 3H), 4.00 (m, 4H), 6.91 (m, 4H), 7.17 (d, 2H), 7.58 (d, 2H), 8.36 (s, 1H), 8.85 (s, 1H).

### Preparative Example 8

### Preparation of Compound (18) in Reaction Scheme 9

### 8-1: 4-{5-[4-(2-methylamino-5-morpholin-4-ylmethyl-thiazol-4-yl)-phenoxy]-pentyloxy}-benzonitrile (18)

30 ml of ethanol was added to 6.50 g (16.5 mmol) of 4-{5-[4-(2-methylamino-thiazol-4-yl)-phenoxy]-pentyloxy}-benzonitrile (20) obtained in Preparative Example 3-1, and 13.7 ml (165 mmol) of formaldehyde (35%) and 14.3 ml (165 mmol) of morpholine were added thereto. The mixture was stirred at 70° C. for 2 hrs. The reaction mixture was cooled to room temperature, diluted with ethyl acetate, and washed with brine. The organic layer was dried over magnesium sulfate, the solvent was removed therefrom, and purified by column chromatography to obtain 860 mg (yield: 11%) of a title compound (33).

¹H-NMR (DMSO-d₆) (ppm) 1.57 (m, 2H), 1.78 (m, 4H), 2.41 (m, 4H), 2.80 (s, 3H), 3.34 (m, 2H), 3.56 (m, 4H), 4.01 (m, 4H), 6.92 (m, 4H), 7.49 (d, 2H), 7.59 (d, 2H).

### Preparative Example 9

### Preparation of Compound (37) in Reaction Scheme 10

### 9-1: 4-{5-[4-(2-amino-thiazol-4-yl)-phenoxy]-pentyloxy}-benzonitrile (33)

22.5 g (55.9 mmol) of 4-{5-[4-(2-bromo-acetyl)-phenoxy]-pentyloxy}-benzonitrile (7) obtained in Preparative Example 1-3 was added to 100 ml of ethanol, and 8.51 g (112 mmol) of thiourea was added thereto. The mixture was refluxed at 80° C. for 12 hrs. The reaction mixture was cooled to room temperature, the solvent was removed therefrom, recrystallized from methanol, and then dried under reduced pressure to obtain 20.7 g (yield: 98%) of a title compound (33).

¹H-NMR (DMSO-d₆) (ppm) 1.57 (m, 2H), 1.78 (m, 4H), 4.06 (m, 4H), 7.04 (d, 2H), 7.09 (d, 2H), 7.10 (s, 1H), 7.64 (d, 2H), 7.75 (d, 2H), 8.90 (brs, 1H).

### 9-2: 4-{5-[4-(2-piperidin-1-yl-thiazol-4-yl)-phenoxy]-pentyloxy}-benzonitrile (35)

15 g (40 mmol) of 4-{5-[4-(2-amino-thiazol-4-yl)-phenoxy]-pentyloxy}-benzonitrile (33) obtained in the above 8-1 was dissolved in 45 ml of dimethylformamide, and 3.5 g (90 mmol) of sodium hydride was slowly added thereto, followed by stirring for 20 min. 6.0 ml (43 mmol) of 1,5-dibromopentane was added thereto, and the mixture was stirred at 55° C.-60° C. for 4 hrs. The reaction mixture was diluted with ethyl acetate, and washed with purified water. The organic layer was dried over magnesium sulfate, the solvent was distilled under reduced pressure, purified by column chromatography and dried under reduced pressure to obtain 12 g (yield: 67%) of a title compound (35).

¹H-NMR (DMSO-d₆) (ppm) 1.57 (m, 2H), 1.63 (m, 6H), 1.77 (m, 4H), 3.60 (m, 4H), 4.04 (m, 4H), 7.04 (d, 4H), 7.10 (s, 1H), 7.40 (d, 2H), 7.68 (d, 2H).

### 9-3: 4-{5-[4-(5-bromo-2-piperidin-1-yl-thiazol-4-yl)-phenoxy]-pentyloxy}-benzonitrile (36)

10 ml of chloroform was added to 250 mg (0.56 mmol) of 4-{5-[4-(2-piperidin-1-yl-thiazol-4-yl)-phenoxy]-pentyloxy}-benzonitrile (35) obtained in the above 8-2, and 0.03 ml (0.67 mmol) of bromine was added thereto, followed by stirring at room temperature for 1 hr. The solvent was removed from the reaction solution, and then the resultant was used as a starting material.

### 9-4: 4-{5-[4-(5-morpholin-4-yl-2-piperidin-1-yl-thiazol-4-yl)-phenoxy]-pentyloxy}-benzonitrile (37)

0.97 ml of morpholine was added to 4-{5-[4-(5-bromo-2-piperidin-1-yl-thiazol-4-yl)-phenoxy]-pentyloxy}-benzonitrile (36) obtained in the above 8-3, and then stirred at 120° C. for 3 hrs. The reaction mixture was cooled to room temperature, diluted with ethyl acetate, and washed with a sodium bicarbonate solution and a sodium chloride solution. The organic layer was dried over anhydrous magnesium sulfate, the solvent was removed therefrom, and purified by column chromatography to obtain 50 mg (yield: 17%) of a title compound (37).

¹H-NMR (DMSO-d₆ (ppm) 1.59 (m, 8H), 1.79 (m, 4H), 2.75 (m, 4H), 3.41 (m, 4H), 3.73 (m, 4H), 4.01 (m, 4H), 6.92 (m, 4H), 7.60 (d, 2H), 7.92 (d, 2H).

### Example 1

### Preparation of N-hydroxy-4-{5-[4-(2-methyl-5-morpholin-4-yl-thiazol-4-yl)-phenoxy]-pentyloxy}-benzamidine (1)

170 mg (0.37 mmol) of 4-{5-[4-(2-methyl-5-morpholin-4-yl-thiazol-4-yl)-phenoxy]-pentyloxy}-benzonitrile (12) obtained in Preparative Example 1-6 was added to 10 ml of ethanol, and 0.10 ml (0.73 mmol) of trimethylamine and 51 mg (0.73 mmol) of hydroxylamine hydrochloride were added thereto. The mixture was refluxed under stirring at 80° C. for 8 hrs. The reaction mixture was distilled under reduced pressure, diluted with ethyl acetate, and washed with purified water and a sodium chloride solution. The organic layer was dried over anhydrous magnesium sulfate, the solvent was distilled under reduced pressure, separated by column chromatography, and dried under reduced pressure to obtain a title compound.

¹H-NMR (DMSO-d₆) (ppm) 1.58 (m, 2H), 1.79 (m, 4H), 2.59 (s, 3H), 2.78 (m, 4H), 3.73 (m, 4H), 4.01 (m, 4H), 5.71 (s, 2H), 6.93-6.98 (m, 4H), 7.58 (d, 2H), 8.05 (d, 2H), 9.45 (s, 1H)

### Examples 2 to 7

The compounds (12) obtained in the same manner as in the Preparative Example 1-6 were prepared in the same manner as Example 1, so as to obtain the title compound (1a).

The used solvents and ¹H-NMR data of the title compounds are shown in Table 1.

TABLE 1

| Example | Chemical name | ¹H-NMR | Solvent |
|---|---|---|---|
| 1 | N-hydroxy-4-{5-[4-(2- | 1.58(m, 2H), 1.79(m, 4H), | DMSO-d₆ |
| | methyl-5-morpholin-4-yl- | 2.59(s, 3H), 2.78(m, 4H), | |
| | thiazol-4-yl)- | 3.73(m, 4H), 4.01(m, 4H), | |
| | phenoxy]-pentyloxy}- | 5.71(s, 2H), 6.93-6.98(m, 4H), | |
| | benzamidine | 7.58(d, 2H), 8.05 (d, 2H), | |
| | | 9.45(s, 1H) | |
| 2 | N-hydroxy-4-(5-{4-[2- | 1.58(m, 2H), 1.80(m, 4H), | DMSO-d₆ |
| | methyl-5-(4-methyl-piperazin- | 2.23(s, 3H), 2.48(m, 4H), | |
| | 1-yl)-thiazol-4-yl]- | 2.58(s, 3H), 2.80(m, 4H), | |
| | phenoxy}-pentyloxy)- | 4.02(m, 4H), 5.71(s, 2H), | |
| | benzamidine | 6.96(m, 4H), 7.59(d, 1H), | |
| | | 7.83(d, 1H), 8.04(d, 2H), | |
| | | 9.44(s, 1H) | |
| 3 | N-hydroxy-4-{5-[4-(2- | 1.58(m, 2H), 1.81(m, 4H), | DMSO-d₆ |
| | amino-5-morpholin-4-yl- | 2.72(m, 4H), 3.17(s, 2H), | |
| | thiazol-4-yl)- | 3.71(m, 4H), 4.03(m, 4H), | |
| | phenoxy]-pentyloxy}- | 6.80(d, 1H), 6.91(d, 1H), | |
| | benzamidine | 6.99(d, 2H), 7.62(d, 2H), | |
| | | 7.83(d, 1H), 8.01(d, 1H) | |
| 4 | N-hydroxy-4-(5-{4-[5-(4- | 1.58(m, 2H), 1.79(m, 4H), | DMSO-d₆ |
| | methyl-piperazin-1-yl)-2- | 2.23(s, 3H), 2.49(m, 4H), | |
| | morpholin-4-yl-thiazol-4-yl]- | 2.77(m, 4H), 3.36(m, 4H), | |
| | phenoxy}-pentyloxy)- | 3.70(m, 4H), 4.02(m, 4H), | |
| | benzamidine | 5.72(s, 2H), 6.94(m, 4H), | |
| | | 7.59(d, 2H), 8.05(d, 2H), | |
| | | 9.45(s, 1H) | |
| 5 | N-hydroxy-4-{5-[4-(2,5- | 1.60(m, 2H), 1.79(m, 4H), | DMSO-d₆ |
| | di-morpholin-4-yl- | 2.75(m, 4H), 3.39(m, 4H), | |
| | thiazol-4-yl)- | 3.72(m, 8H), 4.01(m, 4H), | |
| | phenoxy]-pentyloxy}- | 5.72(s, 2H), 6.93(m, 4H), | |
| | benzamidine | 7.59(d, 2H), 8.06(d, 2H), | |
| | | 9.45(s, 1H) | |
| 6 | N-hydroxy-4-{5-[4-(2- | 1.60(m, 2H), 1.79(m, 4H), | DMSO-d₆ |
| | morpholin-4-yl-5-thiomorpholin- | 2.51(m, 6H), 2.77(m, 2H), | |
| | 4-yl-thiazol-4-yl)- | 2.98(m, 2H), 3.34(m, 4H), | |
| | phenoxy]-pentyloxy}- | 3.70(m, 6H), 4.02(m, 4H), | |
| | benzamidine | 5.72(s, 2H), 6.92(m, 4H), | |
| | | 7.59(d, 2H), 7.77(d, 1H), | |
| | | 8.03(d, 1H), 9.45(s, 1H) | |
| 7 | N-hydroxy-4-{5-[4-(2- | 1.59(m, 2H), 1.80(m, 4H), | DMSO-d₆ |
| | morpholin-4-yl-5-pyrrolidin- | 1.89(m, 4H), 2.91(m, 4H), | |
| | 1-yl-thiazol-4-yl)- | 3.36(m, 4H), 3.70(m, 4H), | |
| | phenoxy]-pentyloxy}- | 4.02(m, 4H), 5.72(s, 2H), | |
| | benzamidine | 6.93(m, 4H), 7.48(d, 2H), | |
| | | 7.94(d, 2H), 9.45(s, 1H) | |

### Examples 8 to 22

The compounds (18) obtained in the same manner as in the Preparative Example 2-5 were prepared in the same manner as Example 1, so as to obtain the title compound (1b).

The used solvents and ¹H-NMR data of the title compounds are shown in Tables 2 and 3.

TABLE 2

| Example | Chemical name | ¹H-NMR | Solvent |
|---|---|---|---|
| 8 | N-hydroxy-4-{5-[4-(2- | 1.59(m, 2H), 1.80(m, 4H), | DMSO-d₆ |
| | methyl-5-morpholin-4- | 2.51(m, 4H), 3.34(s, 3H), | |
| | ylmethyl-thiazol-4-yl)- | 3.61(m, 4H), 3.77(s, 2H), | |
| | phenoxy]-pentyloxy}- | 4.03(m, 4H), 5.71(s, 2H), | |
| | benzamidine | 6.92(d, 2H), 7.01(d, 2H), | |
| | | 7.58(m, 4H), 9.44(s, 1H) | |
| 9 | N-hydroxy-4-(5-{4-[2- | 1.61(m, 2H), 1.81(m, 4H), | DMSO-d₆ |
| | methyl-5-(4-methyl-piperazin- | 2.17(s, 3H), 2.35(m, 4H), | |
| | 1-ylmethyl)-thiazol-4-yl]- | 2.51(m, 4H), 3.33(s, 3H), | |
| | phenoxy}-pentyloxy)- | 3.75(s, 1H), 3.80(s, 1H), | |
| | benzamidine | 4.03(m, 4H), 5.71(s, 2H), | |
| | | 6.92(d, 2H), 7.01(d, 2H), | |
| | | 7.58(m, 4H), 9.44(s, 1H) | |
| 10 | N-hydroxy-4-{5-[4-(2- | 1.59(m, 2H), 1.80(m, 4H), | DMSO-d₆ |
| | methyl-5-thiomorpholin-4- | 2.65(m, 4H), 2.78(m, 4H), | |
| | ylmethyl-thiazol-4-yl)- | 3.33(s, 3H), 3.79(s, 2H), | |
| | phenoxy]-pentyloxy}- | 4.03(m, 4H), 5.71(s, 2H), | |
| | benzamidine | 6.93(d, 2H), 7.00(d, 2H), | |
| | | 7.58(m, 4H), 9.44(s, 1H) | |
| 11 | N-hydroxy-4-{5-[4-(2- | 1.41(m, 2H), 1.53(m, 4H), | DMSO-d₆ |
| | methyl-5-piperidin-1- | 1.59(m, 2H), 1.80(m, 4H), | |
| | ylmethyl-thiazol-4-yl)- | 2.49(m, 4H), 3.33(s, 3H), | |
| | phenoxy]-pentyloxy}- | 3.70(s, 1H), 3.75(s, 1H), | |
| | benzamidine | 4.03(m, 4H), 5.71(s, 2H), | |
| | | 6.92(d, 2H), 7.01(d, 2H), | |
| | | 7.58(m, 4H), 9.44(s, 1H) | |
| 12 | N-hydroxy-4-{5-[4-(5- | 1.59(m, 2H), 1.80(m, 4H), | DMSO-d₆ |
| | dimethylaminomethyl-2- | 2.28(d, 6H), 3.33(s, 3H), | |
| | methyl-thiazol-4-yl)- | 3.69(s, 1H), 3.74(s, 1H), | |
| | phenoxy]-pentyloxy}- | 4.03(m, 4H), 5.71(s, 2H), | |
| | benzamidine | 6.92(d, 2H), 7.01(d, 2H), | |
| | | 7.58(m, 4H), 9.44(s, 1H) | |
| 13 | N-hydroxy-4-{5-[4-(5- | 0.88(t, 3H), 1.32-1.42(m, 4H), | DMSO-d₆ |
| | butylaminomethyl-2- | 1.58(m, 2H), 1.80(m, 4H), | |
| | methyl-thiazol-4-yl)- | 2.48(s, 3H), 2.51(m, 2H), | |
| | phenoxy]-pentyloxy}- | 3.92(s, 2H), 4.02(m, 4H), | |
| | benzamidine | 5.72(s, 2H), 6.91-6.98(m, 4H), | |
| | | 7.55-7.61(m, 4H), 9.46(s, 1H) | |
| 14 | N-hydroxy-4-(5-{4-[5- | 0.89(d, 6H), 1.58(m, 2H), | DMSO-d₆ |
| | (isobutylamino-methyl)-2- | 1.68(m, 1H), 1.80(m, 4H), | |
| | methyl-thiazol-4-yl]- | 2.40(d, 2H), 2.48(s, 3H), | |
| | phenoxy}-pentyloxy)- | 3.91(s, 2H), 4.02(m, 4H), | |
| | benzamidine | 5.72(s, 2H), 6.92(d, 2H), | |
| | | 7.00(d, 2H), 7.55-7.60(m, 4H), | |
| | | 9.45(s, 1H) | |
| 15 | N-hydroxy-4-(5-{4-[5- | 1.08(s, 9H), 1.58(m, 2H), | DMSO-d₆ |
| | (tert-butylamino-methyl)-2- | 1.81(m, 4H), 2.47(s, 3H), | |
| | methyl-thiazol-4-yl]- | 3.88(s, 2H), 4.02(m, 4H), | |
| | phenoxy}-pentyloxy)- | 5.72(s, 2H), 6.93(d, 2H), | |
| | benzamidine | 6.98(d, 2H), 7.55-7.58(m, 4H), | |
| | | 9.46(s, 1H) | |

TABLE 3

| Example | Chemical name | ¹H-NMR | Solvent |
|---|---|---|---|
| 16 | N-hydroxy-4-{5-[4-(2-methyl- | 0.86 (t, 3H), | DMSO-d₆ |
| | 5-propylaminomethyl- | 1.44-1.46 (m, 2H), | |
| | thiazol-4-yl)-phenoxy]- | 1.58 (m, 2H), 1.80 (m, 4H), | |
| | pentyloxy}-benzamidine | 2.48 (s, 3H), 2.51 (m, 2H), | |
| | | 3.91 (s, 2H), 4.02 (m, 4H), | |
| | | 5.72 (s, 2H), 6.92 (d, 2H), | |
| | | 6.97 (d, 2H), | |
| | | 7.55-7.60 (m, 4H), | |
| | | 9.46 (s, 1H) | |
| 17 | N-hydroxy-4-[5-(4-{2-methyl- | 1.58 (m, 2H), 1.80 (m, 4H), | DMSO-d₆ |
| | 5-[(2-morpholin- | 2.35 (m, 2H), 2.40 (m, 4H), | |
| | 4-yl-ethylamino)-methyl]- | 2.48 (s, 3H), 2.70 (m, 2H), | |
| | thiazol-4-yl}-phenoxy)- | 3.56 (m, 4H), 3.95 (s, 2H), | |
| | pentyloxy]-benzamidine | 4.01 (m, 4H), 5.72 (s, 2H), | |
| | | 6.93 (d, 2H), 6.99 (d, 2H), | |
| | | 7.55-7.60 (m, 4H), | |
| | | 9.46 (s, 1H) | |
| 18 | N-hydroxy-4-[5-(4-{5-[(3- | 1.59 (m, 2H), 1.78 (m, 4H), | DMSO-d₆ |
| | imidazol-1-yl-propylamino)- | 1.85 (m, 2H), 2.48 (s, 3H), | |
| | methyl]-2-methyl- | 2.51 (m, 4H), 3.91 (s, 2H), | |
| | thiazol-4-yl-phenoxy]- | 4.02 (m, 4H), 5.72 (s, 2H), | |
| | pentyloxy}-benzamidine | 6.87 (s, 1H), 6.93 (d, 2H), | |
| | | 6.98 (m, 3H), 7.16 (s, 1H), | |
| | | 7.55-7.61 (m, 4H), | |
| | | 9.46 (s, 1H) | |
| 19 | N-hydroxy-4-{5-[4-(2-methyl- | 1.59 (m, 2H), 1.69 (m, 4H), | DMSO-d₆ |
| | 5-pyrrolidin-1-ylmethyl- | 1.80 (m, 4H), 2.49 (m, 4H), | |
| | thiazol-4-yl)- | 2.62 (s, 1.603H), | |
| | phenoxy]-pentyloxy}-benzamidine | 3.79 (s, 2H), 4.03 (m, 4H), | |
| | | 5.73 (s, 2H), | |
| | | 6.93-7.00 (m, 4H), | |
| | | 7.54-7.58 (m, 4H), | |
| | | 9.46 (s, 1H) | |
| 20 | N-hydroxy-4-{5-[4-(5-imidazol- | 1.58 (m, 2H), 1.79 (m, 4H), | DMSO-d₆ |
| | 1-ylmethyl-2-methyl- | 2.48 (s.3H), 4.03 (m, 4H), | |
| | thiazol-4-yl)-phenoxy]- | 5.54 (s, 2H), 5.72 (s, 2H), | |
| | pentyloxy}-benzamidine | 6.94 (m, 3H), 7.02 (d, 2H), | |
| | | 7.29 (s, 1H), | |
| | | 7.56-7.61 (m, 4H), | |
| | | 7.81 (s, 1H), 9.46 (s, 1H) | |
| 21 | N-hydroxy-4-(5-{4-[5-(benzylamino- | 1.58 (m, 2H), 1.79 (m, 4H), | DMSO-d₆ |
| | methyl)-2- | 2.49 (s.3H), 3.78 (s, 2H), | |
| | methyl-thiazol-4-yl]-phenoxy}- | 3.91 (s, 2H), 4.01 (m, 4H), | |
| | pentyloxy)-benzamidine | 5.72 (s, 2H) 6.92 (d, 2H), | |
| | | 7.00 (d, 2H), 7.25 (m, 1H), | |
| | | 7.36 (m, 4H) | |
| | | 7.55-7.61 (m, 4H), | |
| | | 9.46 (s, 1H) | |
| 22 | N-hydroxy-4-{5-[4-(5-cyclopropylaminomethyl- | 0.30 (m, 2H), 0.39 (m, 2H), | DMSO-d₆ |
| | 2-methyl-thiazol-4-yl)- | 1.59 (m, 2H), 1.80 (m, 4H), | |
| | phenoxy]-pentyloxy}-benzamidine | 2.20 (m, 1H), 2.48 (s, 3H), | |
| | | 3.96 (s, 2H), 4.02 (m, 4H), | |
| | | 5.72 (s, 2H), 6.92 (d, 2H), | |
| | | 7.00 (d, 2H), 7.58 (m, 4H), | |
| | | 9.45 (s, 1H) | |

### Example 23

The compounds (22) obtained in the same manner as in the Preparative Example 3-3 were prepared in the same manner as Example 1, so as to obtain the title compound (1c).

The used solvents and ¹H-NMR data of the title compounds are shown in Table 4.

TABLE 4

| Example | Chemical name | ¹H-NMR | Solvent |
|---|---|---|---|
| 23 | N-hydroxy-4-{5- | 1.58 (m, 2H), 1.80 (m, 4H), | DMSO-d₆ |
| | [4-(2-methylamino- | 2.72 (m, 4H), 3.34 (s, 3H), | |
| | 5-morpholin- | 3.71 (m, 4H), 4.01 (m, 4H), | |
| | 4-yl-thiazol-4-yl)- | 5.77 (s, 2H), 6.92 (m, 4H), | |
| | phenoxy]- | 7.59 (d, 2H), 8.05 (d, 2H), | |
| | pentyloxy}- | 9.47 (s, 1H) | |
| | benzamidine | | |

### Examples 24 to 36

The compounds (25) obtained in the same manner as in the Preparative Example 4-2 were prepared in the same manner as Example 1, so as to obtain the title compound (1d).

The used solvents and ¹H-NMR data of the title compounds are shown in Tables 5 and 6.

TABLE 5

| Example | Chemical name | ¹H-NMR | Solvent |
|---|---|---|---|
| 24 | N-hydroxy-4-(5-{4-[2-(methyl- | 1.59 (m, 2H), 1.78 (m, 4H), | DMSO-d₆ |
| | pyridin-4-ylmethyl- | 2.40 (m, 4H), 3.07 (s, 3H), | |
| | amino)-5-morpholin- | 3.34 (m, 2H), 3.56 (m, 4H), | |
| | 4-ylmethyl-thiazol-4- | 4.01 (m, 4H), | |
| | yl]-phenoxy}-pentyloxy)- | 4.73-4.80 (m, 2H), | |
| | benzamidine | 5.72 (brs, 2H), | |
| | | 6.91 (m, 4H), 7.28 (m, 2H), | |
| | | 7.49 (m, 2H), 7.57 (m, 2H), | |
| | | 8.53 (m, 2H), 9.45 (brs, 1H) | |
| 25 | N-hydroxy-4-[5-(4-{2-[(2- | 1.58 (m, 2H), 1.78 (m, 4H), | DMSO-d₆ |
| | hydroxy-ethyl)-methyl- | 2.40 (m, 4H), 3.04 (s, 3H), | |
| | amino]-5-morpholin- | 3.37 (m, 4H), 3.46 (m, 2H), | |
| | 4-ylmethyl-thiazol-4-yl}- | 3.60 (m, 4H), 3.98 (m, 4H), | |
| | phenoxy)-pentyloxy]- | 4.82 (t, 1H), | |
| | benzamidine | 5.72 (brs, 2H), | |
| | | 6.88 (m, 4H), 7.42 (d, 2H), | |
| | | 7.58 (d, 2H), 9.45 (brs, 1H) | |
| 26 | N-hydroxy-4-(5-{4-[2-(ethyl- | 1.12 (t, 3H), 1.58 (m, 2H), | DMSO-d₆ |
| | methyl-amino)-5- | 1.79 (m, 4H), 2.50 (m, 4H), | |
| | morpholin-4-ylmethyl-thiazol- | 2.97 (s, 3H), 3.01 (m, 2H), | |
| | 4-yl]-phenoxy}- | 3.35 (m, 2H), 3.44 (m, 4H), | |
| | pentyloxy)-benzamidine | 3.98 (m, 4H), | |
| | | 5.72 (brs, 2H), | |
| | | 6.89 (m, 4H), 7.42 (d, 2H), | |
| | | 7.58 (d, 2H), 9.46 (brs, 1H) | |
| 27 | N-hydroxy-4-(5-{4-[2-(benzyl- | 1.57-1.60 (m, 2H), | DMSO-d₆ |
| | methyl-amino)-5- | 1.77-1.81 (m, 4H), | |
| | morpholin-4-ylmethyl- | 2.40 (s, 4H), 3.00 (s, 3H), | |
| | thiazol-4-yl]-phenoxy}- | 3.55 (s, 6H), | |
| | pentyloxy)-benzamidine | 4.01-4.03 (m, 4H), | |
| | | 4.68 (s, 2H), 5.71 (s, 2H), | |
| | | 6.92 (d, 2H), 6.98 (d, 2H), | |
| | | 7.28-7.37 (m, 5H), | |
| | | 7.52 (d, 2H), 7.59 (d, 2H), | |
| | | 9.45 (s, 1H) | |
| 28 | N-hydroxy-4-[5-(4-{2-[methyl- | 1.58 (m, 2H), 1.79 (m, 4H), | DMSO-d₆ |
| | (2-morpholin-4- | 2.40 (m, 8H), 2.51 (m, 4H), | |
| | yl-ethyl)-amino]-5-morpholin- | 3.01 (s, 3H), 3.55 (m, 12H), | |
| | 4-ylmethyl-thiazol- | 3.99 (m, 4H), 5.74 (m, 2H), | |
| | 4-yl}-phenoxy)-pentyloxy]- | 6.91 (d, 2H), 6.93 (d, 2H), | |
| | benzamidine | 7.49 (d, 2H), 7.60 (d, 2H), | |
| | | 9.48 (s, 1H) | |
| 29 | N-hydroxy-4-[5-(4-{2-[methyl- | 1.59 (m, 2H), 1.77 (m, 4H), | DMSO-d₆ |
| | (2-morpholin-4- | 2.41 (m, 4H), 2.51 (m, 2H), | |
| | yl-ethyl)-amino]-5-thiomorpholin- | 2.63 (m, 8H), 3.01 (s, 3H), | |
| | 4-ylmethyl- | 3.54 (m, 8H), 4.00 (m, 4H), | |
| | thiazol-4-yl}-phenoxy)- | 5.74 (m, 2H), 6.93 (m, 4H), | |
| | pentyloxy]-benzamidine | 7.47 (d, 2H), 7.61 (d, 2H), | |
| | | 9.49 (s, 1H) | |
| 30 | N-hydroxy-4-[5-(4-{5-{[bis- | 1.58 (m, 2H), 1.80 (m, 4H), | DMSO-d₆ |
| | (2-methoxy-ethyl)- | 2.43 (m, 4H), 2.50 (m, 2H), | |
| | amino]-methyl}-2-[methyl- | 2.63 (m, 2H), 3.01 (s, 3H), | |
| | (2-morpholin-4-yl- | 3.18 (m, 4H), 3.36 (m, 8H), | |
| | ethyl)-amino]-thiazol- | 3.55 (m, 6H), 3.74 (m, 2H), | |
| | 4-yl}-phenoxy)-pentyloxy]- | 4.01 (m, 4H), 5.72 (s, 2H), | |
| | benzamidine | 6.92 (m, 4H) 7.42 (d, 2H), | |
| | | 7.59 (d, 2H), 9.46 (s, 1H) | |

TABLE 6

| Example | Chemical name | ¹H-NMR | Solvent |
|---|---|---|---|
| 31 | N-hydroxy-4-(5-{4-[2-[methyl- | 1.58 (m, 2H), 1.80 (m, 4H), | DMSO-d₆ |
| | (2-morpholin-4- | 2.16 (s, 3H), 2.42 (m, 6H), | |
| | yl-ethyl)-amino]-5-(4- | 3.01 (s, 3H), 3.17 (m, 8H), | |
| | methyl-piperazin-1-ylmethyl)- | 3.55 (m, 8H), 4.01 (m, 4H), | |
| | thiazol-4-yl]-phenoxy}- | 5.71 (m, 2H), 6.94 (m, 4H), | |
| | pentyloxy)-benzamidine | 7.48 (d, 2H), 7.59 (d, 2H), | |
| | | 9.46 (s, 1H) | |
| 32 | N-hydroxy-4-[5-(4-{5-(isopropylamino- | 0.98 (d, 6H), 1.58 (m, 2H), | DMSO-d₆ |
| | methyl)- | 1.80 (m, 4H), 2.42 (m, 4H), | |
| | 2-[methyl-(2-morpholin- | 2.51 (m, 1H), 3.02 (s, 3H), | |
| | 4-yl-ethyl)-amino]-thiazol- | 3.31 (m, 2H), 3.54 (m, 6H), | |
| | 4-yl}-phenoxy)- | 3.79 (m, 2H), 4.00 (m, 4H), | |
| | pentyloxy]-benzamidine | 5.72 (s, 2H), | |
| | | 6.93-6.96 (m, 4H), | |
| | | 7.49 (d, 2H), 7.58 (d, 2H), | |
| | | 8.32 (s, 1H), 9.45 (s, 1H) | |
| 33 | N-hydroxy-4-[5-(4-{5-[(2- | 1.57 (m, 2H), 1.78 (m, 4H), | DMSO-d₆ |
| | methoxy-ethylamino)- | 2.42 (m, 4H), 2.50 (m, 6H), | |
| | methyl]-2-[methyl-(2- | 3.02 (s, 3H), 3.36 (m, 5H), | |
| | morpholin-4-yl-ethyl)- | 3.53 (m, 6H), 4.00 (m, 4H), | |
| | amino]-thiazol-4-yl}-phenoxy)- | 5.71 (brs, 2H), | |
| | pentyloxy]-benzamidine | 6.91 (m, 4H), 7.44 (m, 2H), | |
| | | 7.59 (m, 2H), 9.46 (brs, 1H) | |
| 34 | N-hydroxy-4-[5-(4-{2-[(2- | 1.58 (m, 2H), 1.80 (m, 4H), | DMSO-d₆ |
| | methoxy-ethyl)-methyl- | 2.37 (m, 4H), 3.03 (s, 3H), | |
| | amino]-5-morpholin- | 3.26 (s, 3H), 3.55 (m, 10H), | |
| | 4-ylmethyl-thiazol-4-yl}- | 4.00 (m, 4H), 5.72 (s, 2H), | |
| | phenoxy)-pentyloxy]- | 6.93-6.97 (m, 4H), | |
| | benzamidine | 7.49 (d, 2H), 7.59 (d, 2H), | |
| | | 9.46 (s, 1H). | |
| 35 | N-hydroxy-4-(5-{4-[2-(methyl- | 0.88 (t, 3H), 1.59 (m, 4H), | DMSO-d₆ |
| | propyl-amino)-5- | 1.79 (m, 4H), 2.39 (m, 4H), | |
| | morpholin-4-ylmethyl- | 3.00 (s, 3H), 3.35 (m, 2H), | |
| | thiazol-4-yl]-phenoxy}- | 3.55 (m, 6H), 4.00 (m, 4H), | |
| | pentyloxy)-benzamidine | 5.72 (s, 2H), 6.95 (m, 4H), | |
| | | 7.49 (d, 2H), 7.60 (d, 2H), | |
| | | 9.45 (s, 1H). | |
| 36 | N-hydroxy-4-(5-{4-[2-(methyl- | 1.58 (m, 2H), 1.78 (m, 4H), | DMSO-d₆ |
| | pyridin-3-ylmethyl- | 2.38 (m, 4H), 3.03 (s, 3H), | |
| | amino)-5-morpholin- | 3.56 (m, 6H), 4.01 (m, 4H), | |
| | 4-ylmethyl-thiazol-4- | 4.72 (s, 2H), 5.72 (s, 2H), | |
| | yl]-phenoxy}-pentyloxy)- | 6.92 (d, 2H), 6.98 (d, 2H), | |
| | benzamidine | 7.39 (m, 1H), 7.52 (d, 2H), | |
| | | 7.59 (d, 2H), 7.74 (m, 1H), | |
| | | 8.49 (m, 1H), 8.57 (m, 1H), | |
| | | 9.46 (s, 1H). | |

### Examples 37 to 40

The compounds (29) obtained in the same manner as in the Preparative Example 5-3 were prepared in the same manner as Example 1, so as to obtain the title compound (1e).

The used solvents and ¹H-NMR data of the title compounds are shown in Table 7.

TABLE 7

| Example | Chemical name | ¹H-NMR | Solvent |
|---|---|---|---|
| 37 | N-hydroxy-4-{5-[4-(2-methyl- | 1.58 (m, 2H), 1.79 (m, 4H), | DMSO-d₆ |
| | 5-methylamino-thiazol- | 2.50 (s, 3H), 2.61 (s, 3H), | |
| | 4-yl)-phenoxy]-pentyloxy}- | 4.04 (m, 4H), 5.70 (s, 2H), | |
| | benzamidine | 6.92 (m, 4H), 7.58 (d, 2H), | |
| | | 7.89 (d, 1H), 7.97 (d, 1H), 9.43 (s, 1H) | |
| 38 | N-hydroxy-4-[5-(4-{2-methyl- | 1.20 (m, 2H), 1.84 (m, 4H), | DMSO-d₆ |
| | 5-[(pyridine-4-carbonyl)- | 2.65 (s, 3H), 4.01 (m, 4H), | |
| | amino]-thiazol- | 5.73 (s, 2H), 6.92 (d, 2H), | |
| | 4-yl}-phenoxy)-pentyloxy]- | 7.01 (d, 2H), 7.58 (d, 2H), | |
| | benzamidine | 7.74 (d, 2H), 7.85 (d, 2H), | |
| | | 8.80 (d, 2H), 9.45 (s, 1H) | |
| 39 | N-hydroxy-4-[5-(4-{2-methyl- | 1.58 (m, 2H), 1.78 (m, 4H), | DMSO-d₆ |
| | 5-[(pyridine-3-carbonyl)- | 2.64 (s, 3H), 4.01 (m, 4H), | |
| | amino]-thiazol- | 5.70 (s, 2H), 6.91 (d, 2H), | |
| | 4-yl}-phenoxy)-pentyloxy]- | 7.02 (d, 2H), 7.58 (m, 3H), | |
| | benzamidine | 7.78 (d, 2H), 8.29 (d, 1H), | |
| | | 8.77 (d, 1H), 9.10 (s, 1H), 9.43 (s, 1H) | |
| 40 | N-hydroxy-4-[5-(4-{2-phenyl- | 1.58 (m, 2H), 1.79 (m, 4H), | DMSO-d₆ |
| | 5-[(pyridine-3-carbonyl)- | 4.07 (m, 4H), 7.06 (d, 1H), | |
| | amino]-thiazol- | 7.09 (m, 3H), 7.51 (m, 5H), | |
| | 4-yl}-phenoxy)-pentyloxy]- | 7.87 (m, 3H), 7.97 (d, 2H), | |
| | benzamidine | 8.32 (d, 1H), 8.79 (d, 1H), 9.13 (s, 1H) | |

### Examples 41 to 43

The compounds (30) obtained in the same manner as in the Preparative Example 6-1 were prepared in the same manner as Example 1, so as to obtain the title compound (1f).

The used solvents and ¹H-NMR data of the title compounds are shown in Table 8.

TABLE 8

| Example | Chemical name | ¹H-NMR | Solvent |
|---|---|---|---|
| 41 | N-hydroxy-4-{5-[4-(5-dimethylamino- | 1.58 (m, 2H), 1.79 (m, 4H), | DMSO-d₆ |
| | 2-methyl- | 2.57 (s, 3H), 2.61 (s, 6H), | |
| | thiazol-4-yl)-phenoxy- | 4.00 (m, 4H), 5.70 (s, 2H), | |
| | pentyloxy}-benzamidine | 6.91 (d, 2H), 6.96 (d, 2H), | |
| | | 7.59 (d, 2H), 7.98 (d, 2H), | |
| | | 9.44 (s, 1H) | |
| 42 | N-hydroxy-4-{5-[4-(5-dimethylamino- | 1.58 (m, 2H), 1.79 (m, 4H), | DMSO-d₆ |
| | 2-phenyl- | 2.70 (s, 6H), 4.02 (m, 4H), | |
| | thiazol-4-yl)-phenoxy]- | 5.71 (s, 2H), 6.92 (d, 2H), | |
| | pentyloxy}-benzamidine | 7.01 (d, 2H), 7.47 (m, 3H), | |
| | | 7.60 (d, 2H), 7.89 (d, 2H), | |
| | | 8.04 (d, 2H), 9.46 (s, 1H) | |
| 43 | N-hydroxy-4-{5-[4-(2-cyclohexyl- | 1.23 (m, 1H), 1.41 (m, 4H), | DMSO-d₆ |
| | 5-dimethylamino- | 1.59 (m, 2H), 1.65 (m, 1H), | |
| | thiazol-4-yl)-phenoxy]- | 1.79 (m, 6H), 2.03 (m, 2H), | |
| | pentyloxy}-benzamidine | 2.61 (s, 6H), 2.85 (m, 1H), | |
| | | 4.01 (m, 4H), 5.72 (s, 2H), | |
| | | 6.94 (m, 4H), 7.60 (d, 2H), | |
| | | 7.99 (d, 2H), 9.46 (s, 1H). | |

### Example 44

The compounds (12) obtained in the same manner as in the Preparative Example 7-3 were prepared in the same manner as Example 1, so as to obtain the title compound (1a).

The used solvents and ¹H-NMR data of the title compounds are shown in Table 9.

TABLE 9

| Exam- | | | |
|---|---|---|---|
| ple | Chemical name | ¹H-NMR | Solvent |
| 44 | N-hydroxy-4-{5- | 1.55 (m, 2H), 1.76 (m, 4H), | DMSO-d₆ |
| | [4-(2-methyl- | 2.74 (s, 3H), 3.99 (m, 4H), | |
| | 5-[1,2,4]triazol- | 5.71 (s, 2H), 6.90 (m, 4H), | |
| | 1-yl-thiazol-4-yl)- | 7.17 (d, 2H), 7.58 (d, 2H), | |
| | phenoxy]-pentyloxy}- | 8.36 (s, 1H), 8.84 (s, 1H), | |
| | benzamidine | 9.45 (s, 1H) | |

### Examples 45 to 49

The compounds (27) obtained in the same manner as in the Preparative Example 5-2 were prepared in the same manner as Example 1, so as to obtain the title compound (1g).

The used solvents and ¹H-NMR data of the title compounds are shown in Table 10.

TABLE 10

| Example | Chemical name | ¹H-NMR | Solvent |
|---|---|---|---|
| 45 | N-hydroxy-4-{5-[4-(5-amino- | 1.59 (m, 2H), 1.80 (m, 4H), | DMSO-d₆ |
| | 2-phenyl-thiazol- | 4.02 (m, 4H), 5.73 (s, 2H), | |
| | 4-yl)-phenoxy]-pentyloxy}- | 5.88 (s, 2H), 6.94 (d, 2H), | |
| | benzamidine | 6.98 (d, 2H), 7.40 (t, 1H), | |
| | | 7.42 (d, 2H), 7.59 (d, 2H), | |
| | | 7.74 (d, 2H), 7.76 (d, 2H), 9.45 | |
| | | (s, 1H) | |
| 46 | N-hydroxy-4-{5-[4-(5-amino- | 1.58 (m, 2H), 1.77 (m, 4H), | DMSO-d₆ |
| | 2-methyl-thiazol- | 2.44 (s, 3H), 3.99 (m, 4H), | |
| | 4-yl)-phenoxy]-pentyloxy}- | 5.34 (s, 2H), 5.71 (s, 2H), | |
| | benzamidine | 6.92 (m, 4H), 7.59 (d, 2H), | |
| | | 7.67 (d, 2H), 9.44 (s, 1H) | |
| 47 | N-hydroxy-4-{5-[4-(5-amino- | 1.60 (m, 2H), 1.81 (m, 4H), | DMSO-d₆ |
| | 2-pyridin-3-yl-thiazol- | 4.03 (m, 4H), 5.72 (s, 2H), | |
| | 4-yl)-phenoxy]-pentyloxy}- | 6.05 (s, 2H), 6.92 (d, 2H), | |
| | benzamidine | 7.00 (d, 2H), 7.45 (d, 1H), | |
| | | 7.59 (d, 2H), 7.75 (d, 2H), | |
| | | 8.10 (d, 1H), 8.52 (t, 1H), | |
| | | 8.95 (s, 1H), 9.45 (s, 1H) | |
| 48 | N-hydroxy-4-{5-[4-(5-amino- | 1.23 (t, 3H), 1.58 (m, 2H), | DMSO-d₆ |
| | 2-ethyl-thiazol-4- | 1.78 (m, 4H), 2.78 (q, 2H), | |
| | yl)-phenoxy]-pentyloxy}- | 4.01 (m, 4H), 5.38 (s, 2H), | |
| | benzamidine | 5.72 (s, 2H), 6.93 (d, 4H), | |
| | | 7.59 (d, 2H), 7.67 (d, 2H), | |
| | | 9.46 (s, 1H) | |
| 49 | N-hydroxy-4-{5-[4-(5-amino- | 1.23 (m, 1H), 1.36 (m, 4H), | DMSO-d₆ |
| | 2-cyclohexyl-thiazol- | 1.57 (m, 2H), 1.64 (m, 1H), | |
| | 4-yl)-phenoxy]-pentyloxy}- | 1.81 (m, 6H), 1.98 (m, 2H), | |
| | benzamidine | 2.74 (m, 1H), 4.00 (m, 4H), | |
| | | 5.36 (s, 2H), 5.71 (s, 2H), | |
| | | 6.92 (d, 4H), 7.59 (d, 2H), | |
| | | 7.66 (d, 2H), 9.45 (s, 1H) | |

### Examples 50 and 51

The compounds (18) obtained in the same manner as in the Preparative Example 8-1 were prepared in the same manner as Example 1, so as to obtain the title compound (1b).

The used solvents and ¹H-NMR data of the title compounds are shown in Table 11.

TABLE 11

| Example | Chemical name | ¹H-NMR | Solvent |
|---|---|---|---|
| 50 | N-hydroxy-4-{5-[4-(2-methylamino- | 1.58 (m, 2H), 1.78 (m, 4H), | DMSO-d₆ |
| | 5-morpholin- | 2.41 (m, 4H), 2.80 (s, 3H), | |
| | 4-ylmethyl-thiazol-4- | 3.34 (m, 2H), 3.56 (m, 4H), | |
| | yl)-phenoxy]-pentyloxy}- | 4.01 (m, 4H), | |
| | benzamidine | 5.73 (brs, 2H), | |
| | | 6.92 (m, 4H), 7.36 (m, 1H), | |
| | | 7.49 (d, 2H), 7.59 (d, 2H), | |
| | | 9.46 (brs, 1H) | |
| 51 | N-hydroxy-4-{5-[4-(2-morpholin- | 1.60 (m, 2H), 1.80 (m, 4H), | DMSO-d₆ |
| | 4-yl-5-morpholin- | 2.38 (m, 4H), 3.35 (m, 4H), | |
| | 4-ylmethyl-thiazol- | 3.57 (m, 6H), 3.70 (m, 4H), | |
| | 4-yl)-phenoxy]-pentyloxy}- | 4.03 (m, 4H), 5.72 (s, 2H), | |
| | benzamidine | 7.49 (d, 2H), | |
| | | 7.59 (d, 2H), 9.45 (s, 1H) | |

### Example 52

The compounds (37) obtained in the same manner as in the Preparative Example 9-4 were prepared in the same manner as Example 1, so as to obtain the title compound (1h).

The used solvents and ¹H-NMR data of the title compounds are shown in Table 12.

TABLE 12

| Exam- | | | |
|---|---|---|---|
| ple | Chemical name | ¹H-NMR | Solvent |
| 52 | N-hydroxy-4-{5- | 1.59 (m, 8H), 1.79 (m, 4H), | DMSO-d₆ |
| | [4-(5-morpholin- | 2.75 (m, 4H), 3.41 (m, 4H), | |
| | 4-yl-2-piperidin- | 3.73 (m, 4H), 4.00 (m, 4H), | |
| | 1-yl-thiazol-4-yl)- | 5.72 (s, 2H), 6.92 (m, 4H), | |
| | phenoxy]-pentyloxy}- | 7.59 (d, 2H), 8.06 (d, 2H), | |
| | benzamidine | 9.45 (s, 1H) | |

### Experimental Example 1

### Inhibitory Effects on Osteoclast Differentiation

The effect of the benzamidine derivative of the present invention on osteoclast formation and differentiation process was evaluated via co-culture with an osteoblast.

1-1: Preparation of Cells

a) Preparation of Bone Marrow Cells

Tibia was aseptically ectomized from 6 to 8-week-old male ddY mice to harvest bone marrow cells by using a syringe (21G, Korea Green Cross). The bone marrow cells were suspended in 5 mL of an α-MEM medium (Gibco BRL Co., supplemented with sodium bicarbonate (2.0 g/L), streptomycin (100 mg/L) and penicillin (100,000 unit/mL), filtered and then sterilized). The harvested cells were centrifuged at 600×g for 5 minutes to collect the whole quantity. To remove the red blood cells in the bone marrow cells, 3 mL of Tris HCl (0.83% NH₄Cl, pH 7.5) was added and well mixed. After centrifugation, the numbers of the eukaryotic cells in the harvested bone marrow cells were counted, and then immediately used for a co-culture system.

b) Preparation of Osteoblast

The progenitor bone and the parietal bone were aseptically ectomized from 1 to 2-day-old neonatal ICR mice, washed with a phosphate butter solution (PBS), and treated with a mixed enzyme solution (0.2% collagenase and 0.1% dispase) six to seven times (10, 10, 10, 20, 20 and 20 min), and then 3 to 6 groups of the cells, in which a large volume of cells having osteoblastic characteristics were contained, were intensively collected, and washed with medium (serum-free α-MEM). The washed cells were cultured in the α-MEM medium containing 10% FBS for 2 to 3 days. After sub-culturing, the collected cells were used for this experiment, and diluted to a concentration of 1×10⁶ cells/mL for storage at −70° C.

1-2. Measurement of Osteoclast Differentiation

a) Preparation of Sample

The benzamidine derivative of the present invention was dissolved in a sterile distilled water or ethanol to be diluted to a desired concentration. The final volume of the sample added to the cell culture medium was set at a ratio of 1:1000.

b) Reaction with Sample Via Co-Culture System

The bone marrow cells prepared in the above 1-1 and the osteoblast were co-cultured. Both the bone marrow cells (25,000 cells/cm²) and the osteoblast (10,000 cells/cm²) were plated in a 96-well plate using α-MEM medium containing FBS, and then cultured with the samples to be tested for 7 days. Differentiation factors, such as dexamethasone (10⁻⁷ M) and vitamin D (10⁻⁸ M), were also co-added to the medium from the first day of cultivation. The medium was changed with a fresh media containing a mixture of the samples and the differentiation factors every 2 to 3 days.

c) Evaluation of Osteoclast Differentiation

1) Preparation of TRAP (Tartaric Acid Resistance Alkaline Phosphatase) Staining Solution

TRAP was used as a marker to measure the matured osteoclast in consideration of its characteristics showing a positive reaction to a TRAP staining solution.

The TRAP staining solution was prepared in such the manner that 5 mg of naphthol AS-MS phosphate (sigma N-4875) as a substrate and 25 mg of a coloring agent (Fast Red Violet LB salt) were dissolved in N,N-dimethylformamide (about 0.5 mL). 50 ml of a 0.1 N NaHCO₃ buffer solution containing 50 mM tartaric acid (pH 5.0) was added thereto, and the mixture was stored in a refrigerator prior to use as a staining solution.

2) Staining Method

After culturing the cells for 7 days, the medium was removed from the wells, the cells were once washed with PBS, and then fixed with PBS containing 10% formalin for 2 to 5 min. The cells were fixed again in a mixed solution of ethanol and acetone (1/1) for about 1 min, and dried off. The cells were further treated by the TRAP staining solution for 15 minutes, and washed with water and dried off. The osteoclasts with 3 or more nuclei showing a TRAP-positive reaction were counted under a microscopic examination. Each of tests was confirmed at least three times. The inhibitory effect on osteoclast differentiation of each experimental group, relative to negative controls, was expressed as a percentage (%).

The results are shown in Table 13.

TABLE 13

| | Inhibition |
|---|---|
| | of |
| | osteoclast |
| | differentiation |
| | (%) |
| Example | 1 μm |
| 1 | 94.0 |
| 2 | 78.6 |
| 3 | 73.2 |
| 4 | 100 |
| 5 | 98 |
| 6 | 92 |
| 7 | 58 |
| 8 | 96.4 |
| 9 | 96.4 |
| 10 | 92.9 |
| 11 | 92.3 |
| 12 | 64.9 |
| 13 | 100 |
| 14 | 100 |
| 15 | 100 |
| 16 | 100 |
| 17 | 100 |
| 18 | 100 |
| 19 | 100 |
| 20 | |
| 21 | |
| 22 | |
| 23 | 58.9 |
| 24 | 100 |
| 25 | 100 |
| 26 | 98 |
| 27 | 77 |
| 28 | 100 |
| 29 | 97 |
| 30 | 93 |
| 31 | 88 |
| 32 | 100 |
| 33 | 100 |
| 34 | |
| 35 | |
| 36 | |
| 37 | 83.3 |
| 38 | 78.6 |
| 39 | 65.5 |
| 40 | — |
| 41 | 94 |
| 42 | 49 |
| 43 | |
| 44 | 68.0 |
| 45 | 91 |
| 46 | 88 |
| 47 | 86 |
| 48 | |
| 49 | |
| 50 | 100 |
| 51 | 95 |
| 52 | 91 |

As shown in Table 13, the results indicate that the thiazole derivative-substituted benzamidine derivative of the present invention effectively inhibited the osteoclast differentiation at an extremely low concentration.

### Experimental Example 2

### Cytotoxicity Test

The cytotoxic effect of the benzamidine derivative of the present invention was evaluated by carrying out the experiment described below.

The test substance was diluted in an appropriate solvent at a concentration of 10⁻² M. This substance was diluted in an appropriate culture medium for the cells used in the cytotoxicity test to a concentration of 10⁻⁶M, and loaded into a 96-well plate in 100 μl per well. The cell lines to be used in the cytotoxicity test were plated on a 96-well plate in a dose of 1.0×10⁴ cell/100 μl per well, and cultured for 72 hrs. 25 μl of MTT[3-(4,5-dimethyl-2-thiazolyl)-2,5-diphenyl-2H-tetrazolium bromide] dissolved in PBS (2 mg/mL) were added before 4 hrs of the end of culture. After completion of the reaction, the plates were centrifuged, the medium was discarded and 100 μl of DMSO was added to dissolve formazan. Finally, the absorbance of the developed plates was measured at 540 nm. The survival rates of the cells were expressed as % concentration values in comparison with the control group.

The results are shown in Table 14.

TABLE 14

| | Cell survival rate | | |
|---|---|---|---|
| | (10⁻⁶ M) | | |
| | MC3T3- | | |
| Example | E1 | calvaria | HOS |
| 1 | 85.9 | 91.5 | 105.6 |
| 2 | 91.8 | 87.9 | 107.0 |
| 3 | 94.6 | 97.7 | 89.0 |
| 4 | 89 | 93 | 104 |
| 5 | 93 | 98 | 100 |
| 6 | 90 | 106 | 92 |
| 7 | 99 | 101 | 92 |
| 8 | 94.7 | 90.7 | 102.3 |
| 9 | 98.8 | 92.3 | 95 |
| 10 | 93.4 | 94.5 | 101 |
| 11 | 91.9 | 95.8 | 101.9 |
| 12 | 98.8 | 95.6 | 107.5 |
| 13 | 93 | 96 | 106 |
| 14 | 98 | 103 | 111 |
| 15 | 99 | 105 | 98 |
| 16 | 92 | 95 | 100 |
| 17 | 103 | 93 | 99 |
| 18 | 99 | 94 | 106 |
| 19 | 103 | 97 | 90 |
| 23 | 92.0 | 95.8 | 104.0 |
| 24 | 86 | 95 | 112 |
| 25 | 100 | 86 | 86 |
| 26 | 98 | 107 | 102 |
| 27 | 102 | 106 | 104 |
| 28 | 11 | 104 | 114 |
| 29 | 109 | 99 | 109 |
| 30 | 105 | 103 | 107 |
| 31 | 110 | 102 | 101 |
| 32 | 104 | 98 | 106 |
| 33 | 104 | 94 | 110 |
| 37 | 94.3 | 92.5 | 105.3 |
| 38 | 100.0 | 94.6 | 117.6 |
| 39 | 95.0 | 86.3 | 92.1 |
| 41 | 91 | 93 | 107 |
| 42 | 91 | 98 | 100 |
| 45 | 81 | 83 | 104 |
| 46 | 90 | 95 | 106 |
| 47 | 97 | 99 | 112 |
| 50 | 91 | 90 | 102 |
| 51 | 88 | 102 | 91 |
| 52 | 94 | 97 | 97 |

As shown in Table 14, the results indicate that the benzamidine derivative of the present invention shows little cytotoxicity.

Hereinbelow, Formulation Example for the composition of the present invention will be described.

### Formulation Example

### Pharmaceutical Preparation

### 1. Preparation of Powders

| | |
|---|---|
| Benzamidine derivative of Formula 1 | 2 g |
| Lactose | 1 g |

The above-components were mixed, and then filled into an air tight bag to prepare a powder.

### 2. Preparation of Tablets

| | |
|---|---|
| Benzamidine derivative of Formula 1 | 100 mg |
| Corn starch | 100 mg |
| Lactose | 100 mg |
| Magnesium stearate | 2 mg |

The above-components were mixed, and then tabletted with a common tabletting method to prepare a tablet.

### 3. Preparation of Capsules

| | |
|---|---|
| Benzamidine derivative of Formula 1 | 100 mg |
| Corn starch | 100 mg |
| Lactose | 100 mg |
| Magnesium stearate | 2 mg |

The above components were mixed, and then filled into a gelatin capsule according to a common preparation method of capsule to prepare a capsule.

### 4. Preparation of Injections

| Benzamidine derivative of Formula 1 | 10 | μg/ml |
|---|---|---|
| Dilute hydrochloric acid BP | to pH 3.5 | |
| Injectable NaCl BP | max. 1 | ml |

The benzamidine derivative of Formula 1 was dissolved in an adequate volume of injectable sodium chloride BP, then pH of the resulting solution was controlled to pH 3.5 with dilute hydrochloric acid BP. The volume of the solution was adjusted with injectable sodium chloride BP, and the solution was mixed fully. The solution was filled into a type I ampoule (5 ml) made with transparent glass, and then the ampoule was sealed under the upper air lattice by melting glass. The sealed ampoule was autoclaved at 120° C. for 15 minutes or longer for sterilization to prepare an injection.

1. A benzamidine derivative represented by the following Formula 1 or a pharmaceutically acceptable salt thereof: 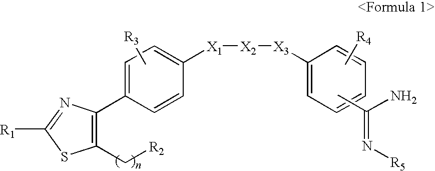
wherein R₁ is methyl, ethyl, isopropyl, phenyl, pyridinyl, cyclohexyl, morpholinyl, 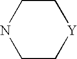
which is unsubstituted or substituted with C₁˜C₆ alkyl, NR₆R₇ or CH₂NR₆R₇;
R₂ is a primary or secondary amine, which is NR₈R₉, 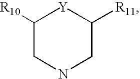
piperidine, pyrrolidine, imidazole or triazole;
R₃ and R₄ are each independently hydrogen, methyl, ethyl, halogen, hydroxy or methoxy group;
R₅ is a hydroxy group;
R₆ and R₇ are each independently hydrogen, methyl, ethyl, propyl, hydroxyethyl, methoxyethyl, 2-morpholinoethyl, benzyl, pyridin-3-ylmethyl, pyridin-4-ylmethyl, 3-pyridinylcarbonyl or ethanesulfonyl;
R₈ and R₉ are each independently hydrogen; methyl; ethyl; propyl; isopropyl; butyl; isobutyl; t-butyl; hydroxyethyl; methoxyethyl; 2-morpholinoethyl; benzyl; 3-imidazol-1-yl-propyl; cyclopropyl; or carbonyl substituted with one group selected from 3-pyridinyl and 4-pyridinyl;
R₁₀ and R₁₁ are each independently hydrogen or methyl;
X₁ and X₃ are each independently oxygen, sulfur, amine or methylamine group;
X₂ is propylene, butylene, pentylene, hexylene, ethylene-O-ethylene, ethylene-NH-ethylene, butylene carbonyl, 2-butenyl, methylene-1,2-phenylene-methylene, methylene-1,3-phenylene-methylene, methylene-1,4-phenylene-methylene or methylene-pyridinyl-methylene;
Y is O, S or methylamino or CH₂ group; and
n is an integer of 0 or 1.

2. The benzamidine derivative or the pharmaceutically acceptable salt thereof according to claim 1, wherein the compound of Formula 1 is selected from the group consisting of:
1) N-hydroxy-4-{5-[4-(2-methyl-5-morpholin-4-yl-thiazol-4-yl)-phenoxy]-pentyloxy}-benzamidine,
2) N-hydroxy-4-(5-{4-[2-methyl-5-(4-methyl-piperazin-1-yl)-thiazol-4-yl]-phenoxy}-pentyloxy)-benzamidine,
3) N-hydroxy-4-{5-[(4-(2-amino-5-morpholin-4-yl-thiazol-4-yl)-phenoxy]-pentyloxy}-benzamidine,
4) N-hydroxy-4-{5-[(4-[5-(4-methyl-piperazin-1-yl)-2-morpholin-4-yl-thiazol-4-yl]-phenoxy}-pentyloxy)-benzamidine,
5) N-hydroxy-4-{5-[4-(2,5-di-morpholin-4-yl-thiazol-4-yl)-phen oxy]-pentyloxy}-benzamidine,
6) N-hydroxy-4-{5-[4-(2-morpholin-4-yl-5-thiomorpholin-4-yl-thiazol-4-yl)-phenoxy]-pentyloxy}-benzamidine,
7) N-hydroxy-4-{5-[4-(2-morpholin-4-yl-5-pyrrolidin-1-yl-thiazol-4-yl)-phenoxy]-pentyloxy}-benzamidine,
8) N-hydroxy-4-{5-[4-(2-methyl-5-morpholin-4-ylmethyl-thiazol-4-yl)-phenoxy]-pentyloxy}-benzamidine,
9) N-hydroxy-4-(5-{4-[2-methyl-5-(4-methyl-piperazin-1-ylmethyl)-thiazol-4-yl]-phenoxy}-pentyloxy)-benzamidine,
10) N-hydroxy-4-{5-[4-(2-methyl-5-thiomorpholin-4-ylmethyl-thiazol-4-yl)-phenoxy]-pentyloxy}-benzamidine,
11) N-hydroxy-4-{5-[4-(2-methyl-5-piperidin-1-ylmethyl-thiazol-4-yl)-phenoxy]-pentyloxy}-benzamidine,
12) N-hydroxy-4-{5-[4-(5-dimethylaminomethyl-2-methyl-thiazol-4-yl)-phenoxy]-pentyloxy}-benzamidine,
13) N-hydroxy-4-{5-[4-(5-butylaminomethyl-2-methyl-thiazol-4-yl)-phenoxy]-pentyloxy}-benzamidine,
14) N-hydroxy-4-(5-{4-[5-(isobutylamino-methyl)-2-methyl-thiazol-4-yl]-phenoxy}-pentyloxy)-benzamidine,
15) N-hydroxy-4-(5-{4-[5-(tert-butylamino-methyl)-2-methyl-thiazol-4-yl]-phenoxy}-pentyloxy)-benzamidine,
16) N-hydroxy-4-{5-[4-(2-methyl-5-propylaminomethyl-thiazol-4-yl)-phenoxy]-pentyloxy}-benzamidine,
17) N-hydroxy-4-[5-(4-{2-methyl-5-[(2-morpholin-4-yl-ethylamino)-methyl]-thiazol-4-yl}-phenoxy)-pentyloxy]-benzamidine,
18) N-hydroxy-4-[5-(4-{5-[(3-imidazol-1-yl-propylamino)-methyl]-2-methyl-thiazol-4-yl}-phenoxy)-pentyloxy]-benzamidine,
19) N-hydroxy-4-{5-[4-(2-methyl-5-pyrrolidin-1-ylmethyl-thiazol-4-yl)-phenoxy]-pentyloxy}-benzamidine,
20) N-hydroxy-4-{5-[4-(5-imidazol-1-ylmethyl-2-methyl-thiazol-4-yl)-phenoxy]-pentyloxy}-benzamidine,
21) N-hydroxy-4-(5-{4-[5-(benzylamino-methyl)-2-methyl-thiazol-4-yl-phenoxy]-pentyloxy}-benzamidine,
22) N-hydroxy-4-{5-[4-(5-cyclopropylaminomethyl-2-methyl-thiazol-4-yl)-phenoxy]-pentyloxy}-benzamidine,
23) N-hydroxy-4-{5-[4-(2-methylamino-5-morpholin-4-yl-thiazol-4-yl)-phenoxy]-pentyloxy}-benzamidine,
24) N-hydroxy-4-(5-{4-[2-(methyl-pyridin-4-ylmethyl-amino)-5-morpholin-4-ylmethyl-thiazol-4-yl]-phenoxy}-pentyloxy)-benzamidine,
25) N-hydroxy-4-[5-(4-{2-[(2-hydroxy-ethyl)-methyl-amino]-5-morpholin-4-ylmethyl-thiazol-4-yl}-phenoxy)-pentyloxy]-benzamidine,
26) N-hydroxy-4-(5-{(4-[2-(ethyl-methyl-amino)-5-morpholin-4-ylmethyl-thiazol-4-yl]-phenoxy}-pentyloxy)-benzamidine,
27) N-hydroxy-4-(5-{4-[2-(benzyl-methyl-amino)-5-morpholin-4-yl methyl-thiazol-4-yl]-phenoxy}-pentyloxy)-benzamidine,
28) N-hydroxy-4-[5-(4-{2-[methyl-(2-morpholin-4-yl-ethyl)-amino]-5-morpholin-4-ylmethyl-thiazol-4-yl}-phenoxy)-pentyloxy]-benzamidine,
29) N-hydroxy-4-[5-(4-{2-[methyl-(2-morpholin-4-yl-ethyl)-amino]-5-thiomorpholin-4-ylmethyl-thiazol-4-yl}-phenoxy)-pentyloxy]-benzamidine,
30) N-hydroxy-4-[5-(4-{5-{[bis-(2-methoxy-ethyl)-amino]-methyl}-2-[methyl-(2-morpholin-4-yl-ethyl)-amino]-thiazol-4-yl}-phenoxy)-pentyloxy]-benzamidine,
31) N-hydroxy-4-(5-{4-[2-[methyl-(2-morpholin-4-yl-ethyl)-amino]-5-(4-methyl-piperazin-1-ylmethyl)-thiazol-4-yl]-phenoxy}-pentyloxy)-benzamidine,
32) N-hydroxy-4-[(5-(4-{5-(isopropylamino-methyl)-2-[methyl-(2-morpholin-4-yl-ethyl)-amino]-thiazol-4-yl}-phenoxy)-pentyloxy]-benzamidine,
33) N-hydroxy-4-[5-(4-{5-[(2-methoxy-ethylamino)-methyl]-2-[methyl-(2-morpholin-4-yl-ethyl)-amino]-thiazol-4-yl}-phenoxy)-pentyloxy]-benzamidine,
34) N-hydroxy-4-[5-(4-{2-[(2-methoxy-ethyl)-methyl-amino]-5-morpholin-4-ylmethyl-thiazol-4-yl}-phenoxy)-pentyloxy]-benzamidine,
35) N-hydroxy-4-(5-{4-[2-(methyl-propyl-amino)-5-morpholin-4-yl methyl-thiazol-4-yl]-phenoxy}-pentyloxy)-benzamidine,
36) N-hydroxy-4-(5-{4-[2-(methyl-pyridin-3-ylmethyl-amino)-5-morpholin-4-ylmethyl-thiazol-4-yl]-phenoxy}-pentyloxy)-benzamidine,
37) N-hydroxy-4-{5-[4-(2-methyl-5-methylamino-thiazol-4-yl)-phenoxy]-pentyloxy}-benzamidine,
38) N-hydroxy-4-[5-(4-{2-methyl-5-[(pyridine-4-carbonyl)-amino]-thiazol-4-yl}-phenoxy)-pentyloxy]-benzamidine,
39) N-hydroxy-4-[5-(4-{2-methyl-5-[(pyridine-3-carbonyl)-amino]-thiazol-4-yl}-phenoxy)-pentyloxy]-benzamidine,
40) N-hydroxy-4-[5-(4-{(2-phenyl-5-[(pyridine-3-carbonyl)-amino]-thiazol-4-yl}-phenoxy)-pentyloxy]-benzamidine,
41) N-hydroxy-4-{5-[4-(5-dimethylamino-2-methyl-thiazol-4-yl)-phenoxy-pentyloxy}-benzamidine,
42) N-hydroxy-4-{5-[4-(5-dimethylamino-2-phenyl-thiazol-4-yl)-phenoxy]-pentyloxy}-benzamidine,
43) N-hydroxy-4-{5-[4-(2-cyclohexyl-5-dimethylamino-thiazol-4-yl)-phenoxy]-pentyloxy}-benzamidine,
44) N-hydroxy-4-{5-[4-(2-methyl-5-[1,2,4]triazol-1-yl-thiazol-4-yl)-phenoxy]-pentyloxy}-benzamidine,
45) N-hydroxy-4-{5-[4-(5-amino-2-phenyl-thiazol-4-yl)-phenoxy]-pentyloxy}-benzamidine,
46) N-hydroxy-4-{5-[4-(5-amino-2-methyl-thiazol-4-yl)-phenoxy]-pentyloxy}-benzamidine,
47) N-hydroxy-4-{5-[4-(5-amino-2-pyridin-3-yl-thiazol-4-yl)-phenoxy]-pentyloxy}-benzamidine,
48) N-hydroxy-4-{5-[4-(5-amino-2-ethyl-thiazol-4-yl)-phenoxy]-pentyloxy}-benzamidine,
49) N-hydroxy-4-{5-[4-(5-amino-2-cyclohexyl-thiazol-4-yl)-phenoxy]-pentyloxy}-benzamidine,
50) N-hydroxy-4-{5-[4-(2-methylamino-5-morpholin-4-ylmethyl-thiazol-4-yl)-phenoxy]-pentyloxy}-benzamidine,
51) N-hydroxy-4-{5-[4-(2-morpholin-4-yl-5-morpholin-4-ylmethyl-thiazol-4-yl)-phenoxy]-pentyloxy}-benzamidine, and
52) N-hydroxy-4-{5-[4-(5-morpholin-4-yl-2-piperidin-1-yl-thiazol-4-yl)-phenoxy]-pentyloxy}-benzamidine.

3. The benzamidine derivative or the pharmaceutically acceptable salt thereof according to claim 1 or 2, wherein the pharmaceutically acceptable salt thereof is hydrochloride or methane sulfonate.

4. A method for preparing a benzamidine derivative of the following Formula 1a, or a pharmaceutically acceptable salt thereof, comprising the steps of 1) to 7):
1) reacting a compound of Formula 2 with a compound of Formula 3 in the presence of an inorganic base to prepare a compound of Formula 4,
2) reacting a compound of Formula 5 with the compound of Formula 4 obtained in step 1) in the presence of an inorganic base to prepare a compound of Formula 6,
3) reacting the compound of Formula 6 obtained in step 2) with a bromine compound to prepare a benzonitrile derivative of Formula 7,
4) reacting the alpha-brominated compound of Formula 7 obtained in step 3) with a thioamide compound of Formula 8 to prepare a benzonitrile derivative having a thiazole group of Formula 9,
5) reacting the compound of Formula 9 obtained in step 4) with a bromine compound to prepare a benzonitrile derivative having a brominated thiazole group of Formula 10,
6) reacting the compound of Formula 10 obtained in step with a primary or secondary amine compound of Formula 11 to prepare a benzonitrile derivative of Formula 12, and
7) reacting the compound of Formula 12 obtained in step 6) with a hydroxylamine or hydrochloric alcohol solution and ammonia to prepare a benzamidine derivative of Formula 1a or a pharmaceutically acceptable salt thereof: 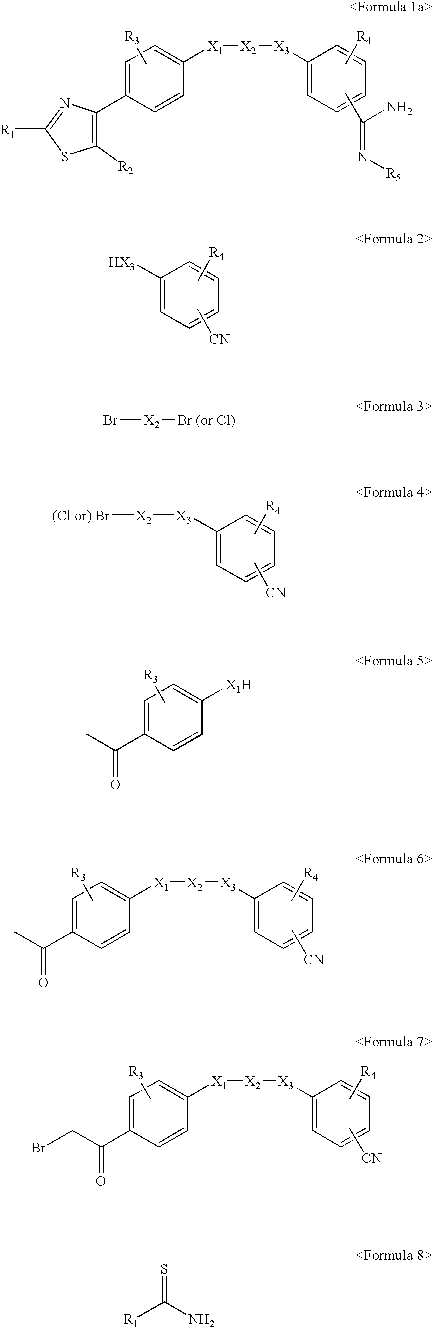 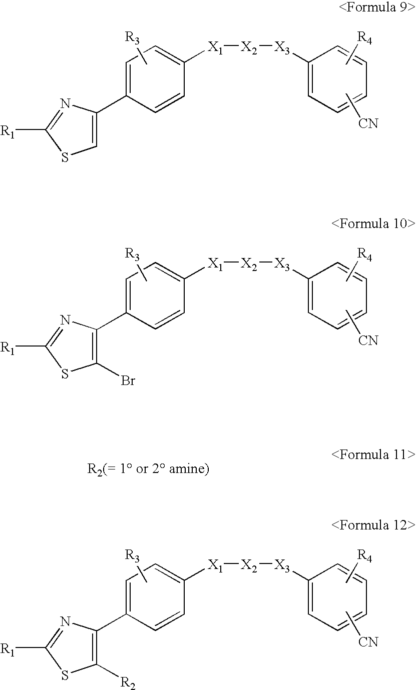
wherein R₁ is methyl, ethyl, isopropyl, phenyl, morpholinyl or amino, and R₂, R₃, R₄, R₅, X₁, X₂ and X₃ are the same as defined in claim 1.

5. A method for preparing a benzamidine derivative of the following Formula 1b, or a pharmaceutically acceptable salt thereof, comprising the steps of 1) to 6):
1) reacting the compound of Formula 4 obtained in step 1) of claim 4 with a compound of Formula 13 to prepare a benzonitrile derivative of Formula 14,
2) reacting the compound of Formula 14 obtained in step 1) with a bromine compound to prepare an alpha-brominated benzonitrile derivative of Formula 15,
3) reacting the alpha-brominated compound of Formula 15 obtained in step 2) with a thioamide compound of Formula 8 to prepare a benzonitrile derivative having a thiazole group of Formula 16,
4) reacting the compound of Formula 16 obtained in step 3) with a bromine compound to prepare a benzonitrile derivative having a brominated thiazole group of Formula 17,
5) reacting the compound of Formula 17 obtained in step 4) with the primary or secondary amine compound of Formula 11 to prepare a benzonitrile derivative of Formula 18, and
6) reacting the compound of Formula 18 obtained in step 5) with a hydroxylamine or hydrochloric alcohol solution and ammonia to prepare a benzamidine derivative of Formula 1b or a pharmaceutically acceptable salt thereof: 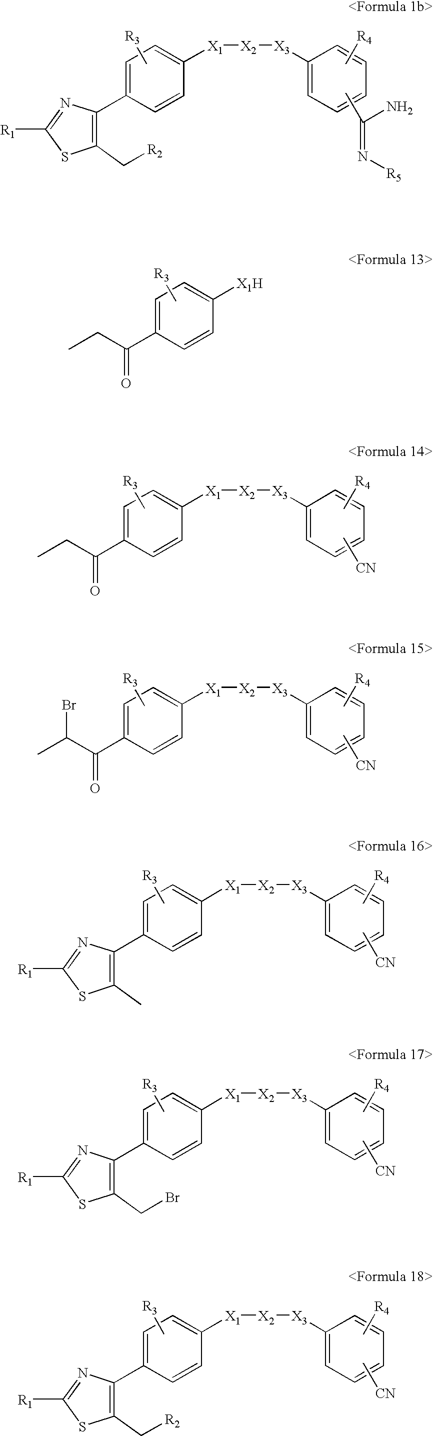
wherein R₁ is methyl, ethyl, isopropyl, or phenyl, and R₂, R₃, R₄, R₅, X₁, X₂ and X₃ are the same as defined in claim 1.

6. A method for preparing a benzamidine derivative of the following Formula 1c, or a pharmaceutically acceptable salt thereof, comprising the steps of 1) to 4):
1) reacting the compound of Formula 7 obtained in step 3) of claim 4 with a thiourea compound (19) to prepare a benzonitrile derivative having an amino-thiazole group of Formula 20,
2) reacting the compound of Formula 20 obtained in step 1) with a bromine compound to prepare a benzonitrile derivative having a brominated amino-thiazole group of Formula 21,
3) reacting the compound of Formula 21 obtained in step 2) with the primary or secondary amine compound of Formula 11 to prepare a benzonitrile derivative of Formula 22, and
4) reacting the compound of Formula 22 obtained in step 3) with a hydroxylamine or hydrochloric alcohol solution and ammonia to prepare a benzamidine derivative of Formula 1c a pharmaceutically acceptable salt thereof: 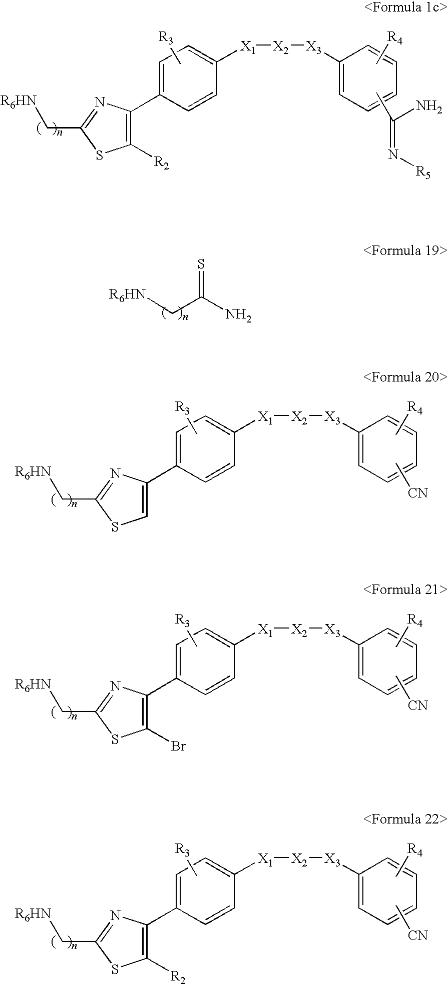
wherein R₂, R₃, R₄, R₅, R₆, X₁, X₂, X₃ and n are the same as defined in claim 1.

7. A method for preparing a benzamidine derivative of the following Formula 1d, or a pharmaceutically acceptable salt thereof, comprising the steps of 1) to 3):
1) reacting the compound of Formula 20 obtained in step 1) of claim 6 with a compound of Formula 23 to prepare a benzonitrile derivative having a thiazole group of Formula 24,
2) reacting the compound of Formula 24 obtained in step 1) with formaldehyde and the primary or secondary amine compound of Formula 11 to prepare a benzonitrile derivative of Formula 25, and
3) reacting the compound of Formula 25 obtained in step 2) with a hydroxylamine or hydrochloric alcohol solution and ammonia to prepare a benzamidine derivative of Formula 1d or a pharmaceutically acceptable salt thereof: 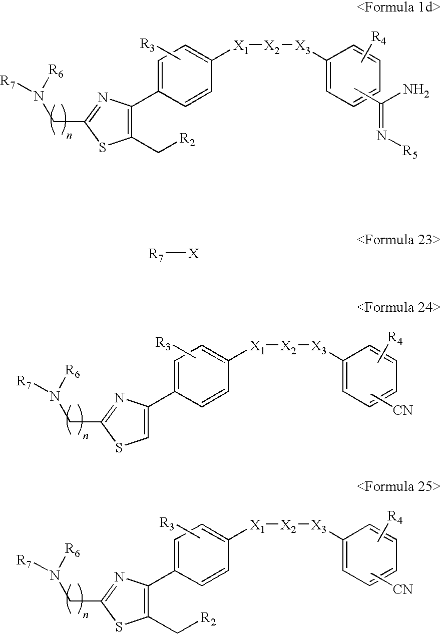
wherein R₂, R₃, R₄, R₅, R₆, R₇, X₁, X₂, X₃ and n are the same as defined in claim 1.

8. A method for preparing a benzamidine derivative of the following Formula 1e, or a pharmaceutically acceptable salt thereof, comprising the steps of 1) to 4):
1) reacting the compound of Formula 9 obtained in step 4) of claim 4 with nitric acid to prepare a benzonitrile derivative having a thiazole group containing a nitrous acid group of Formula 26,
2) reacting the compound of Formula 26 obtained in step 1) with iron or tin chloride dihydrate to prepare a benzonitrile derivative having an amino-thiazole group of Formula 27,
3) reacting the compound of Formula 27 obtained in step 2) with a halide compound of Formula 28 to prepare a benzonitrile derivative substituted with a primary amine of Formula 29, and
4) reacting the compound of Formula 29 obtained in step 3) with a hydroxylamine or hydrochloric alcohol solution and ammonia to prepare a benzamidine derivative of Formula 1e or a pharmaceutically acceptable salt thereof: 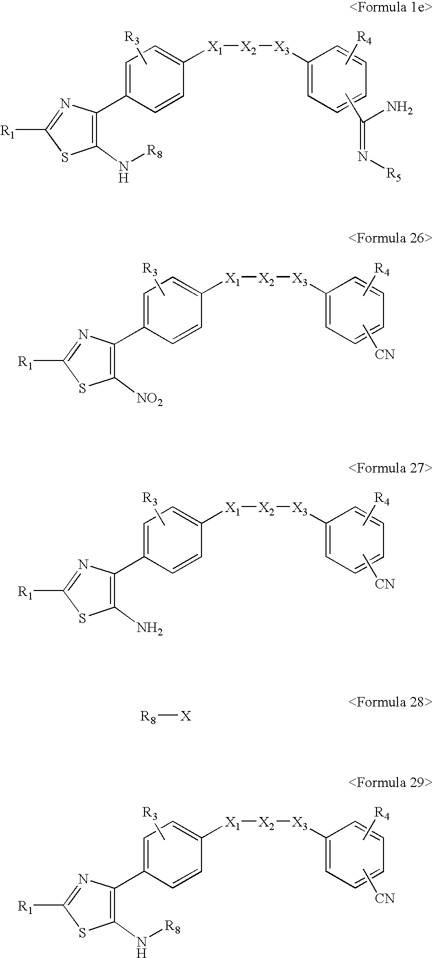
wherein R₁ is methyl, ethyl, isopropyl, phenyl, pyridinyl, or cyclohexyl, and R₃, R₄, R₅, R₈ (except that R₈ is hydrogen), X₁, X₂, and X₃ are the same as defined in claim 1.

9. A method for preparing a benzamidine derivative of the following Formula 1f, or a pharmaceutically acceptable salt thereof, comprising the steps of 1) and 2):
1) reacting the compound of Formula 27 obtained in step 2) of claim 8 with a halide compound of Formula 28 to prepare a benzonitrile derivative substituted with a secondary amine of Formula 30, and
2) reacting the compound of Formula 30 obtained in step 1) with a hydroxylamine or hydrochloric alcohol solution and ammonia to prepare a benzamidine derivative of Formula 1f or a pharmaceutically acceptable salt thereof: 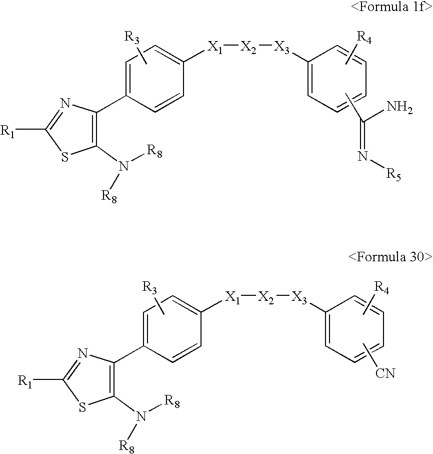
wherein R₁ is methyl, ethyl, isopropyl, phenyl, pyridinyl, or cyclohexyl, and R₃, R₄, R₅, R₈ (except that R₈ is hydrogen), X₁, X₂, and X₃ are the same as defined in claim 1.

10. A method for preparing a benzamidine derivative of the following Formula 1a, or a pharmaceutically acceptable salt thereof, comprising the steps of 1) and 4):
1) reacting the compound of Formula 7 obtained in step 3) of claim 4 with a primary or secondary amine of Formula 11 to prepare a benzonitrile derivative of Formula 31, and
2) reacting the compound of Formula 31 obtained in step 1) with a bromine compound to prepare an alpha-brominated compound of Formula 32,
3) reacting the compound of Formula 32 obtained in step 2) with the thioamide compound of Formula 8 to prepare a benzonitrile derivative having a thiazole group of Formula 12, and
4) reacting the benzonitrile derivative of Formula 12 obtained in step 3) with a hydroxylamine or hydrochloric alcohol solution and ammonia to prepare the benzamidine derivative of Formula 1a or a pharmaceutically acceptable salt thereof: 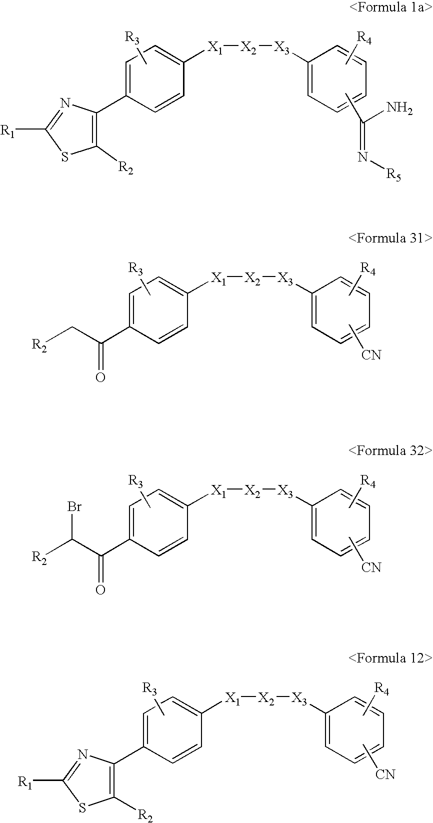
wherein R₁ is methyl, ethyl, isopropyl, or phenyl, and R₂, R₃, R₄, R₅, X₁, X₂ and X₃ are the same as defined in claim 1.

11. A method for preparing a benzamidine derivative of the following Formula 1g, or a pharmaceutically acceptable salt thereof, comprising the step of 1):
1) reacting the compound of Formula 27 obtained in step 2) of claim 8 with a hydroxylamine or hydrochloric alcohol solution and ammonia to prepare a benzamidine derivative of Formula 1g or a pharmaceutically acceptable salt thereof: 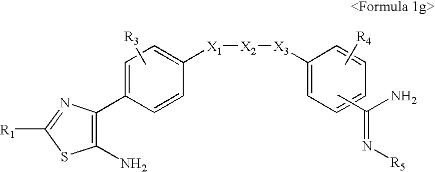
wherein R₁ is methyl, ethyl, isopropyl, phenyl, pyridinyl, or cyclohexyl, and R₃, R₄, R₅, X₁, X₂ and X₃ are the same as defined in claim 1.

12. A method for preparing a benzamidine derivative of the following Formula 1b, or a pharmaceutically acceptable salt thereof, comprising the steps of 1) and 2):
1) reacting the compound of Formula 9 obtained in step 4) of claim 4 with formaldehyde and a primary or secondary amine compound of Formula 11 to prepare a benzonitrile derivative of Formula 18, and
2) reacting the compound of Formula 18 obtained in step 1) with a hydroxylamine or hydrochloric alcohol solution and ammonia to prepare a benzamidine derivative of Formula 1b or a pharmaceutically acceptable salt thereof: 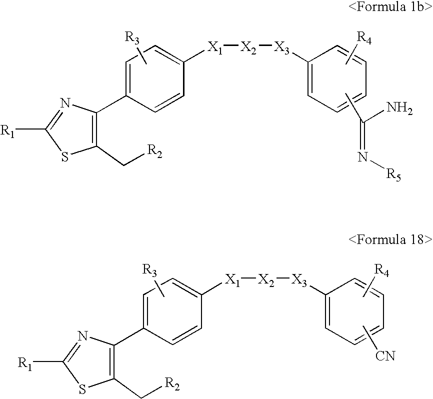
wherein R₁ is 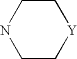
which is unsubstituted or substituted with C₁˜C₆ alkyl, CH₂NR₆R₇ or NR₆R₇ (except that both R₆ and R₇ are hydrogen), and R₂, R₃, R₄, R₅, R₆, R₇, X₁, X₂, X₃ and Y are the same as defined in claim 1.

13. A method for preparing a benzamidine derivative of the following Formula 1h, or a pharmaceutically acceptable salt thereof, comprising the steps of 1) and 5):
1) reacting the compound of Formula 7 obtained in step 3) of claim 4 with a thiourea compound to prepare a benzonitrile derivative having an amino-thiazole group of Formula 33,
2) reacting the compound of Formula 33 obtained in step 1) with a compound of Formula 34, of which both terminals are substituted with halogen, to prepare a benzonitrile derivative of Formula 35 with a thiazole group, which is substituted with a heteroring,
3) reacting the compound of Formula 35 obtained in step 2) with a bromine compound to prepare a benzonitrile derivative having a brominated amino-thiazole group of Formula 36,
4) reacting the compound of Formula 36 obtained in step 3) with the primary or secondary amine compound of Formula 11 to prepare a benzonitrile derivative of Formula 37, and
5) reacting the compound of Formula 37 obtained in step 4) with a hydroxylamine or hydrochloric alcohol solution and ammonia to prepare a benzamidine derivative of Formula 1h or a pharmaceutically acceptable salt thereof: 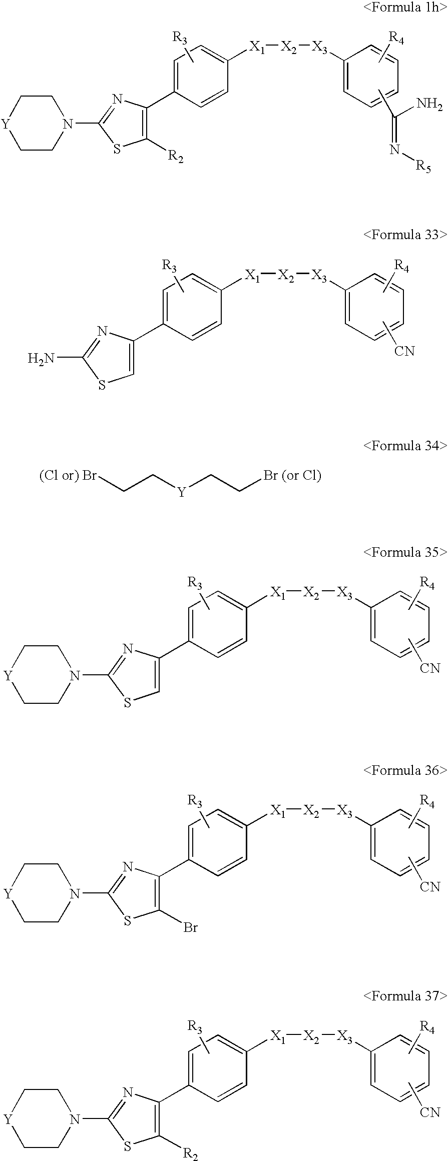
wherein R₂, R₃, R₄, R₅, X₁, X₂, X₃ and Y are the same as defined in claim 1.

14. The method for preparing the benzamidine derivative, or the pharmaceutically acceptable salt thereof according to claim 4, 5, or 10, wherein the thioamide compound of Formula 8 is selected from the group consisting of thioacetamide, thiopropionamide, thioisobutyramide, trimethylthioacetamide, thiohexanoamide, cyclohexancarbothioicacidamide, piperidine-4-carbothioicacidamide, morpholin-4-carbothioicacidamide, N-methylthiourea, N-ethylthiourea, and N-propylthiourea.

15. The method for preparing the benzamidine derivative, or the pharmaceutically acceptable salt thereof according to any one of claims 7 to 9, wherein the halide compound of Formula 23 or 28 is selected from the group consisting of iodomethane, iodoethane, iodopropane, propyl bromide, 2-chloroethyl methyl ether, chloro ethyl morpholine, 3-bromo methylpyridine, bromo ethanol, benzyl bromide, nicotinoyl chloride, ethanesulfonyl chloride and isonicotinoyl chloride.

16. The method for preparing the benzamidine derivative or the pharmaceutically acceptable salt thereof according to any one of claims 4 to 13, wherein in the step of converting benzonitrile into benzamidine, in the case of R₅═OH, the amine to be used is hydroxylamine hydrochloride; and the hydroxylamine hydrochloride is reacted in the presence of a base, with the base being selected from the group consisting of organic bases such as triethylamine, 1,8-diazabicyclo[5.4.0]undec-7-ene (DBU), diethylmethylamine (Et₂NMe), N-methylmorpholine, N-methylpiperidine, pyridine and 2,6-dimethylpyridine, and inorganic bases such as potassium carbonate, potassium bicarbonate, sodium hydroxide, potassium hydroxide, sodium amide, sodium hydride, sodium methoxide, and sodium ethoxide, at 60 to 80° C. for 1 to 9 hrs in a single solvent selected from the group consisting of methanol, ethanol and acetonitrile, or a mixed solvent thereof with water.

17. A pharmaceutical composition for the prevention and treatment of osteoporosis, comprising the compound or the pharmaceutically acceptable salt thereof according to claim 1 or 2.
